(19) United States
(12) Patent Application Publication
(10) Pub. No.: US 2020/0318810 A1
(43) Pub. Date: Oct. 8, 2020

(54) VARIABLE LIGHT SOURCE

(71) Applicant: CamPlex, Inc., Germantown, TN (US)

(72) Inventors: John Tesar, Tucson, AZ (US); Patrick Marcus, Tucson, AZ (US)

(21) Appl. No.: 16/677,441

(22) Filed: Nov. 7, 2019

Related U.S. Application Data
(60) Provisional application No. 62/757,096, filed on Nov. 7, 2018.

(51) Int. Cl. F21V 9/40; F21V 9/20; F21V 8/00; A61B 90/30

(52) U.S. Cl. CPC F21V 9/40; F21V 9/20; F21Y 2115/10; A61B 90/30; G02B 6/0006

(57) ABSTRACT

An illumination device can include a plurality of light sources, a first beam combiner, and a plurality of filters. The plurality of light sources can include first and second light sources configured to generate first and second light beams respectively. The first beam combiner can be configured to receive light from the first and second light sources and combine the first and second light beams to generate a composite light beam. The plurality of filters can include a first filter configured to controllably adjust a spectral power distribution of the first light beam incident on the first filter and a second filter configured to controllably adjust a spectral power distribution of the second light beam incident on the second filter. The first and second filters can be configured to be tuned to alter the spectral power distribution of the first and second light beams.

### CROSS-REFERENCE TO RELATED APPLICATIONS

This application claims the benefit of priority to U.S. Provisional Appl. No. 62/757,096 filed Nov. 7, 2018. The entirety of each application referenced in this paragraph is incorporated herein by reference.

### BACKGROUND

### Field

This disclosure relates generally to light sources such as for surgical visualization. This light source may be adjustable so as to provide the desired illumination. The light source may comprise one or more solid-state light sources such as LEDs and/or laser diodes, which may potentially be coupled to fiber optics (or light pipes) in some cases.

### Description of the Related Art

Surgical visualization systems can assist healthcare providers visualize a surgical site during surgery. Such surgical visualization systems may include one or more types of cameras. Illumination can also be provided to the surgical site to enhance viewing and to assist in the visualization of surgical sites. Additionally, the spectral distribution of such illumination may be suitably tuned to enhance visualization. Such illumination may potentially be varied in different circumstances. Visualization systems may include cameras including but not limited to cameras that provide surgical microscope views, endoscopes, cameras on retractors, cameras on surgical tools, proximal cameras, exoscopes, etc. The visualization systems may include binocular displays that may include one or more displays (e.g., monitors) and may be configured to provide 2D or 3D viewing.

### SUMMARY

Various examples described herein include light sources that can provide light that is directed to a surgical site (e.g., via fiber optics or light pipes). The light source can be a variable light source that can be adjusted to provide different spectral distributions. The light source can therefore be tuned to provide the desired type of lighting.

The light source may include one or more solid state emitters such as Light Emitting Diodes (LEDs) and/or lasers (e.g., laser diodes). These LEDs may be white LEDs and/or color LED's such as red green and blue LEDs. Other colors LEDs as well as other types of light sources may be employed. The light from the light sources can be combined to provide an aggregate beam that is used to illuminate the surgical site.

Light from the one or more light sources can be tuned with a tunable filter that tailors the spectral distribution. Such tunable filters may comprise a filter such as an interference filter that is tilted to alter the spectral characteristics of the filter. Some designs can utilize controllable tilting plates with dichroic coatings applied to their surface(s) such that unwanted peaks from solid state lighting (SSL) sources can be reduced or shifted to more closely conform the light output of a desired illuminant, such as D65, D55, D50 or other desired output.

Light from the various emitters may be passed through different tunable filters to control the spectral distribution of the contributions of light from the different emitters. In this manner, the aggregate beam that is directed onto the surgical site may be tailored to provide the desired spectral characteristics. In some implementations, the use of dichroic filters to superimpose collections of sources over each other by the passing of a wave group through the filter and when the filter is placed at a 45 degree angle passes another differing wave band group. This superposition of waveband outputs can be gathered with fiber assemblies. Superimposing sources can improve the efficiency by reducing the Area of the source, the A in the radiometric calculation of the A*Omega product (where Omega is the solid angle resulting from the source).

Optical fiber may be employed at some stage to deliver the light to the surgical site. For example, the light from the emitters, tuned by the tunable filters and combined may be coupled into fiber optics. Light propagated through the fiber optics maybe directed onto the surgical site. In some designs, the light from the emitters can be gathered by optical coupling from rectangular emitters and the optical fibers or light guides can transmit the flux, tuned by the tunable filters and combined, may be coupled into fiber optics to a more distal position for transmission to the surgical site, or to optical assemblies such as lenses, for further shaping the flux as it is directed to the surgical site.

Accordingly, certain examples described herein include efficient, high-intensity, solid-state light sources such as LEDs and/or laser diodes that provide light that is spectrally tuned and collected and directed possibly into optical fiber or other optics that collects the light. Example designs may thus provide variable light rendering using small high-intensity light sources that propagate light through a collection of pathways for illuminating the receiving end of a fiber optics illumination conduit or light conduit. More particularly, certain embodiments may provide variable light rendering using small high-intensity light sources that output light that is directed to one or more variable filters via a collection of pathways for illuminating one end of a fiber optics illumination conduit or light conduit. Certain examples combine phosphor-coated LED high-intensity light sources and colored light sources (e.g., laser diodes), or phosphor assemblies activated by laser diodes, for excitation via a collection of pathways for illuminating one end of a fiber optics illumination conduit or light conduit.

Various designs may be configured to provide the user with choices for the illumination modes. The light source, for example, may be adjusted by the user to provide different types of illumination having different spectral make-up. The optical spectrum of the light provided may, for example, be adjusted by controlling which light sources is used to provide light as well as possibly by tuning light from the one or more light sources using the tunable filters.

Various designs may include a communication system to receive instructions from the user to control the illumination mode and/or to control the spectral distribution of the light output by the light source. A communication system may also provide communication to one or more displays and/or one or more cameras.

Various designs include an illumination device comprising at least one sub-source comprising a plurality of light emitters configured to produce light flux. The illumination device further comprises a plurality of optical fibers, each optical fiber of the plurality of optical fibers comprising a first end portion configured to receive the light flux from a corresponding light emitter and a second end portion configured to emit the received light flux. The light emitters are arranged in a first pattern, the first end portions are arranged in the first pattern, and the second end portions are arranged in a second pattern different from the first pattern.

Various implementations can include a distributed surgical lighting system comprising a plurality of light emitters, a processor, and a photodetector. The light emitters can be configured to produce a spectral output. The processor can be configured to control an electrical signal from a power source to drive different light emitters to emit light. The photodetector can be configured to measure a spectral contribution of at least one of the plurality of light emitters when other light emitters in the plurality of light emitters are not emitting light.

Various implementations can include a method to determine a spectral contribution of one or more of a plurality of light emitters in real time. The method can include (a) emitting light from one or more light emitters when other light emitters in the plurality are not emitting light and (b) measuring a spectral output of the one or more light emitters emitting light via a photodetector. The method can include repeating (a) and (b) for another one or more light emitters.

Some implementations can include a surgical visualization system comprising at least one camera configured to image a surgical site. The at least one camera can comprise a first illumination source. The surgical visualization system can also include a second illumination source. Each of the first and second illumination sources can be configured to emit light to a surgical site imaged by the at least one camera.

### BRIEF DESCRIPTION OF THE DRAWINGS

FIG. 1A is a schematic diagram of an example spectral filter such as a bandpass interference filter comprising a plurality of layers disposed on a substrate.

FIG. 1B is a schematic illustration of a plot of the optical transmission versus wavelength (both in arbitrary units) for the filter of FIG. 1A showing a peak in optical transmission.

FIG. 1C is a schematic diagram of the spectral filter of FIG. 1A tilted clockwise.

FIG. 1D is a schematic illustration of a plot of the optical transmission versus wavelength (both in arbitrary units) for the filter of FIG. 1A that is tilted thereby resulting in a shift in the peak in optical transmission.

FIG. 1E is a schematic diagram of the spectral filter of FIGS. 1A and 1C further tilted.

FIG. 1F is a schematic illustration of a plot of the optical transmission versus wavelength (both in arbitrary units) for the filter of FIGS. 1A and 1C that is further tilted thereby resulting in a further shift in the peak in optical transmission.

FIG. 1G is a schematic diagram of the spectral filter of FIGS. 1A, 1C, and 1E tilted in the opposite direction (counter clockwise) than the tilt (clockwise) shown in FIGS. 1C and 1E.

FIG. 1H schematically illustrates an example color mixing assembly comprising a plurality of filters in accordance with certain embodiments described herein.

FIG. 1I schematically illustrates another example color mixing assembly comprising a plurality of filters in accordance with certain embodiments described herein.

FIG. 1J schematically illustrates example portions of example color mixing assemblies in accordance with certain embodiments described herein.

FIG. 2A is a block diagram of an example light source comprising a plurality of LEDs including, e.g., white light LEDs, and color LEDs, as well as laser diodes.

FIG. 2B schematically illustrates a portion of an example illumination device in accordance with certain embodiments described herein.

FIGS. 3-6 schematically illustrate example illumination modes that can be offered to the user, for example, via a user interface. Such a user interface may communicate, for example, with one or more displays and one or more cameras.

FIG. 3 illustrates an example Mode A of the light source of FIG. 2A configured to output an approximation of D65 light.

FIG. 4 illustrates an example Mode B of the light source of FIG. 2A configured to output narrow band illumination.

FIG. 5 illustrates an example Mode C of the light source of FIG. 2A configured to output an approximation of D65 light and near IR excitation.

FIG. 6 illustrates an example Mode D of the light source of FIG. 2A configured to output D light excitation.

FIG. 7 schematically illustrates an example optical or wavelength multiplexing assembly/method to aggregate light from multiple light sources.

FIG. 8 schematically illustrates an example spatial multiplexing assembly/method to aggregate light from multiple light sources.

FIGS. 9 and 10 schematically illustrate an example of aggregated light sources providing flux to tilting plates.

FIG. 11 schematically illustrates an example light aggregating system integrated into a fiber optic delivery cable.

FIG. 12 schematically illustrates an example system using wavelength multiplexing for excitation laser sources.

FIG. 13 schematically illustrates an example optical system for imaging a surgical site incorporating a plurality of illumination outputs such as from separate optical fiber outputs or fiber optic multiplexers. The illumination outputs are shown spaced apart and on opposite sides of left and right optical channels of the stereo surgical microscope.

FIG. 14 schematically illustrates a top down view of example multiplexers configured to provide illumination in an optical system for imaging a surgical site.

FIG. 15 schematically illustrates an example multiplexers configured to provide illumination in an optical system for imaging a surgical site.

FIG. 16 schematically illustrates an example multiplexing assembly/method.

FIG. 17 schematically illustrates an example optical system for imaging a surgical site that can benefit from utilizing a distributed fiber optic or light guide assembly having a plurality of outputs. The illumination outputs are shown spaced apart and on opposite sides of left and right optical channels of the stereo surgical microscope.

FIGS. 18 and 19 schematically illustrate the side view of the example optical system for imaging a surgical site shown in FIG. 17.

### DETAILED DESCRIPTION

Surgery is often performed with multiple surgical visualization aids such as, e.g., head worn loupes, microscopes, endoscopes, etc. with each imaging modality potentially being accompanied by an illumination source. As surgeries have become more complex, minimally invasive procedures more common, and cameras less expensive and smaller, surgeons seek an illumination scheme appropriate for a mix of cameras. It can be desirable for the character of the light to be tailored for the camera in use, and the transition from one camera view to another to be neither jarring from the point of view of intensity or color quality irrespective of the camera type.

Various examples described herein include light sources that can provide light that is directed to a surgical site. These light sources may be used to illuminate a surgical site while one or more cameras capture images such as video images of the surgical site and may be used for diagnosis, general medical lighting, or other procedures or activities. Cameras and systems for imaging the surgical site may include, for example, surgical microscope view cameras, endoscopes, cameras on retractors, cameras on surgical tools, proximal cameras, exoscopes, etc. Such surgical visualization systems may display images such as video using a binocular display assembly that include displays that provide views of images obtained from the camera or cameras. The surgical visualization systems may switch from viewing an image or video input from one camera to another or show multiple views simultaneously. Moreover, the light source may be automatically varied when switching from displaying a view from one camera to a view from another camera.

Illumination may facilitate enhanced visualization of the surgical site such as obtained by video camera. Light, for example, can be provided to the surgical site via optical fiber. In some cases where an endoscope is employed to capture images within the body, the light may be provided to the surgical site via the endoscope. In certain embodiments, the light can be provided via fiber optics in various ways. For example, fiber optics can be integrated into an endoscope configured to be inserted into a body via a natural opening or lumen in the body, or through a surgically induced opening in the body. For another example, fiber optics can be integrated into an exoscope (e.g., an imaging device that “stands off” from the patient and provides a surgery site view) or a camera providing surgical microscope views. For another example, fiber optics can be brought near the patient to supplement overhead surgical lighting (e.g., used by the physician sans optical devices or with non-illuminated magnification devices, such as loupes, or to supplement other medical imaging modalities such as endoscopes, exoscopes, or cameras providing surgical microscope views).

The light source can be a variable light source that can be adjusted to provide different spectral distributions. The light source can therefore be tuned to provide the desired type of lighting. In particular, it can be advantageous to provide a light source having a transition (e.g. gradual transition) or a variable change of the output spectral power distribution such as a gradual transition or a variable change between a white light rendering and narrow band imaging.

Certain examples described herein provide such light sources utilizing filters that have a spectral characteristic that varies with orientation such as tilt. Interference filters, for example, have a spectral response, such as spectral transmission or refection that varies with angle of orientation. Accordingly, by varying the orientation of these filters, the spectral distribution of the light interacting with the filter can be adjusted or tuned.

In certain embodiments, the light source can operate in three modes, as well as in combinations of these three modes. The three modes for certain embodiments can be described as follows:

Mode 1: White light for general medical or surgical illumination (e.g., “surface” based illumination). This mode can include the ability to illuminate a scene in a D65-near-equilavent matter, and can include the ability to modify the color temperature of the “white light” (e.g., adjusting the wavelength range of the white light to make it warmer or cooler).

Mode 2: Specialized “surface” based illumination (e.g., short wavelengths; blue waveband; green waveband; D-light; light compatible with photodynamic diagnosis (PDD); light which helps visualize changes in the epithelium, in either of two or more sub-modes). For example, this mode can include a sub-mode comprising activating both blue and green channels in a manner suitable for narrow band imaging (NBI), and can include a sub-mode comprising activating only blue light for a D-light mode.

Mode 3: “Deep penetrating” illumination comprising near infrared (NIR) illumination or excitation. This mode can include either or both of two sub-modes: a sub-mode with a broader-based NIR illumination to reveal below the surface structures in tissue, and a sub-mode with a narrow NIR illumination to excite a dye or other material, which can be used in conjunction with blocking filters.

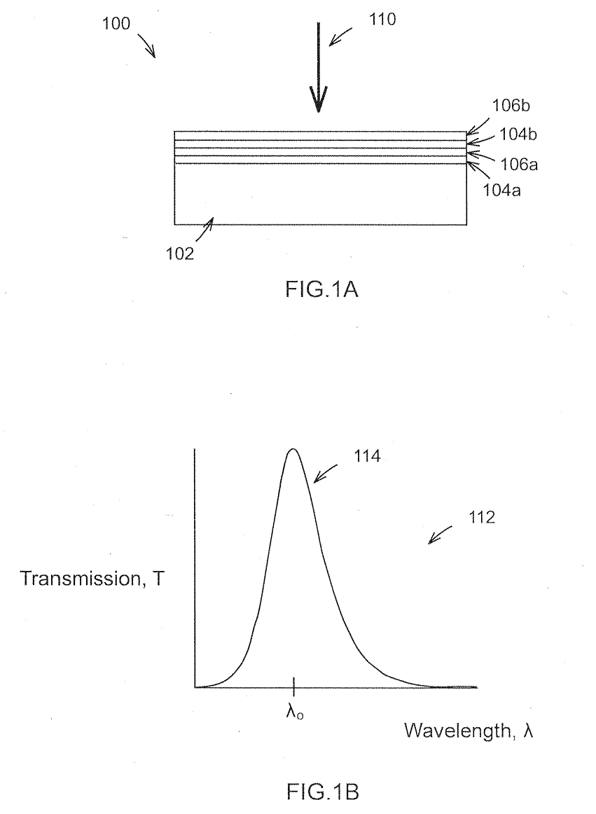

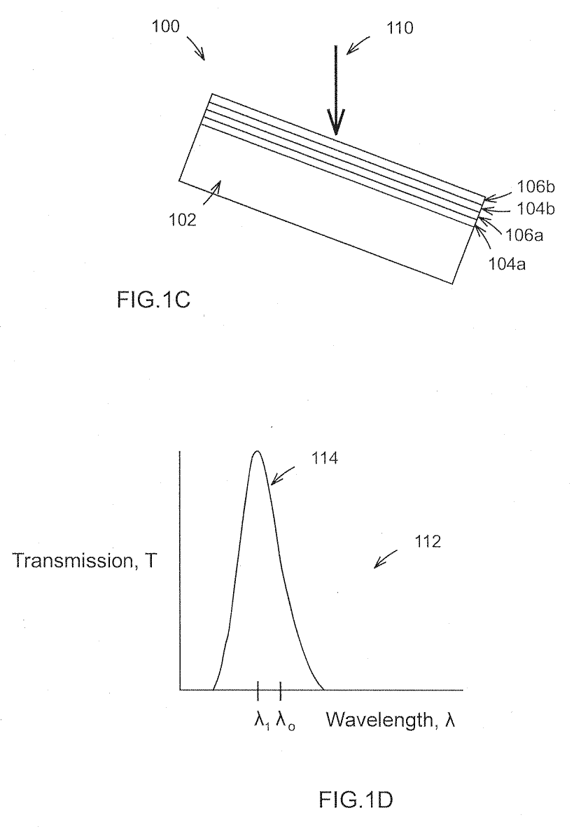

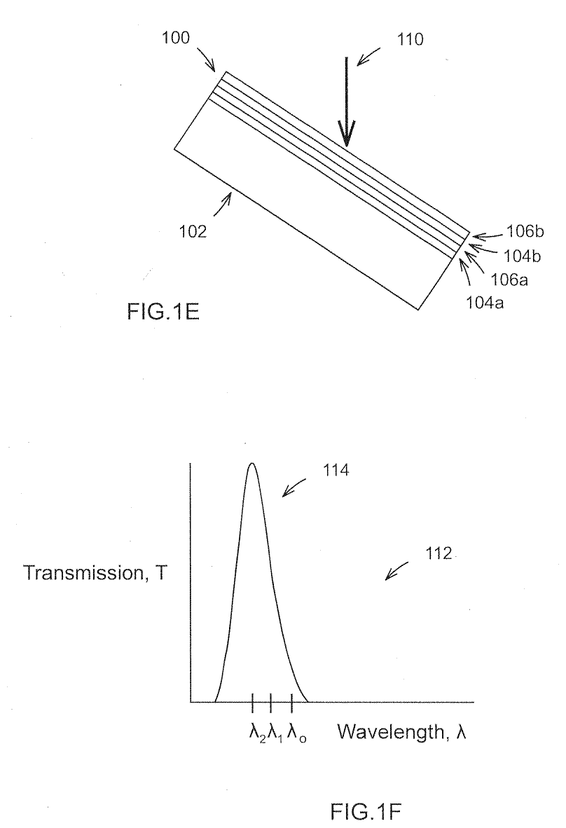

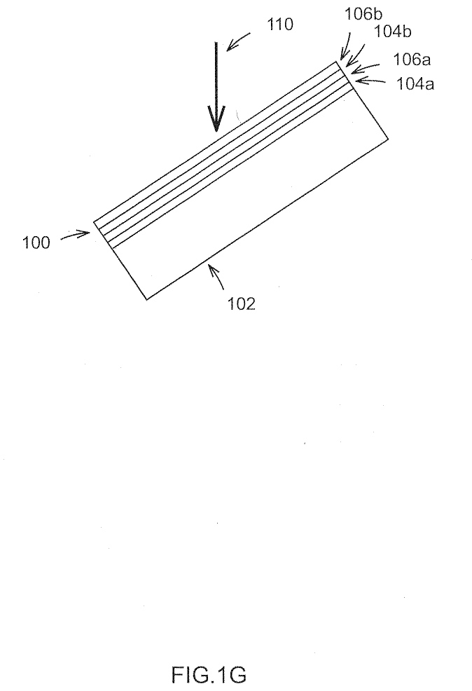

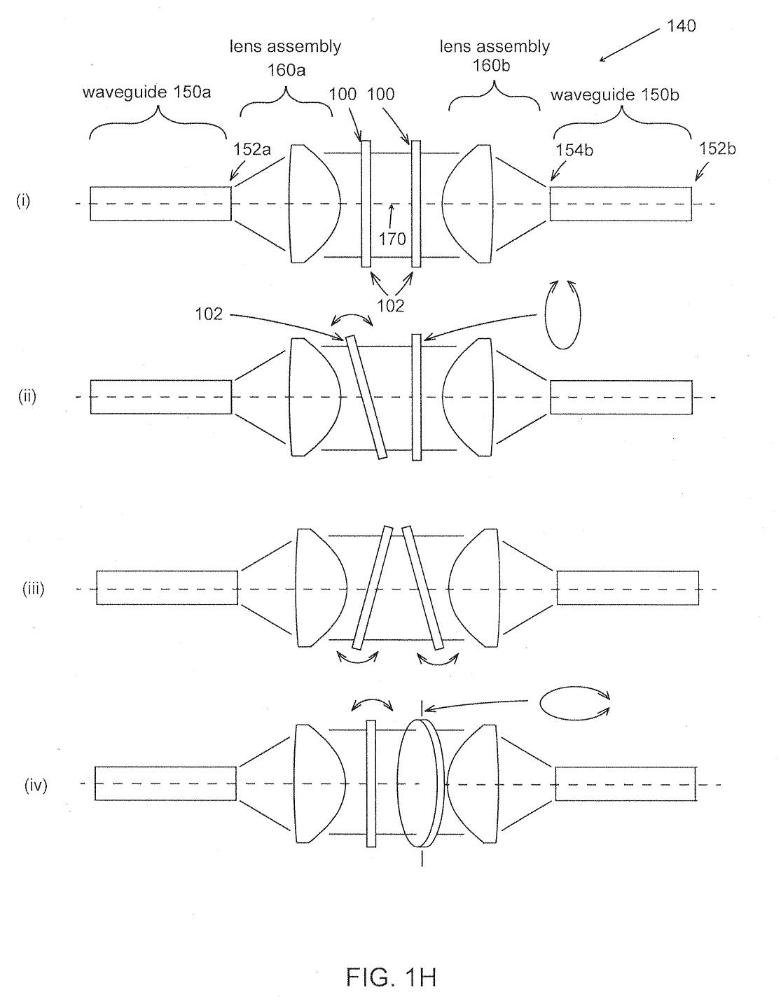

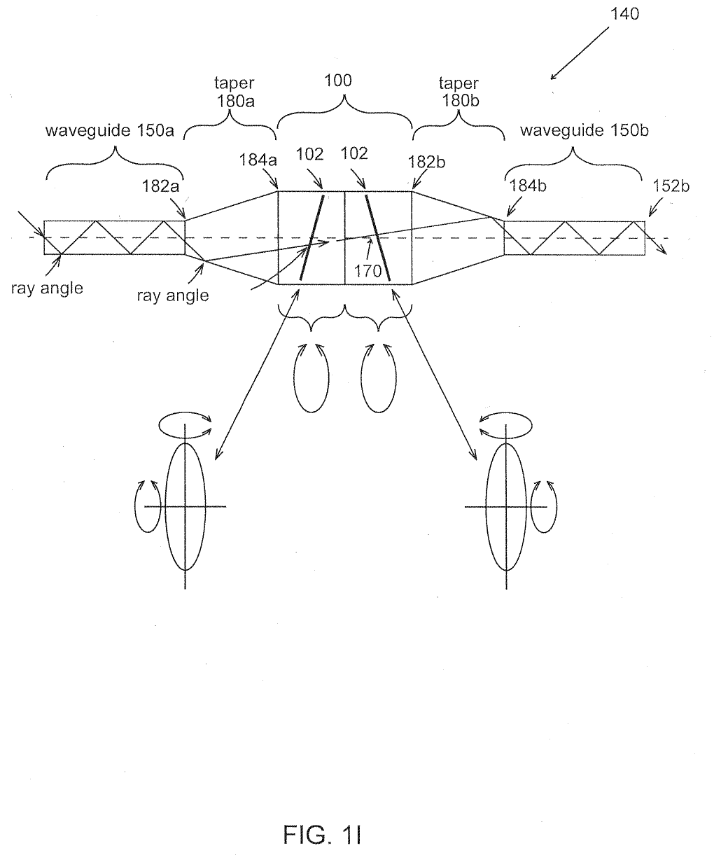

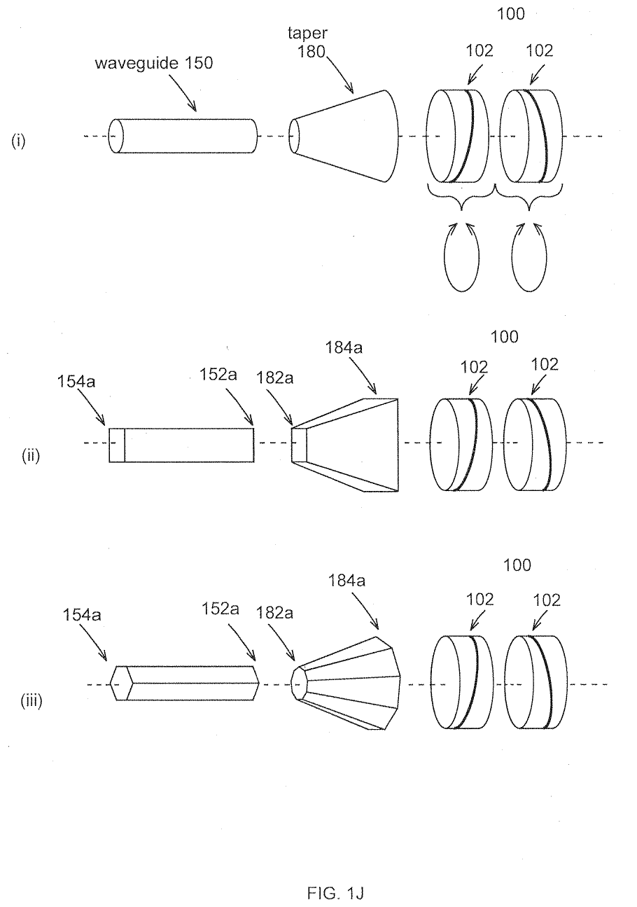

FIG. 1A schematically illustrates a filter 100 such as an interference filter that can be tilted to alter the spectral characteristics of the filter. The filter 100 comprises a substrate 102 on which a plurality of layers 104a, 104b, 106a 106b are disposed. In some designs, a portion of the layers 104a, 104b may comprise a first material and a portion of the layers 106a 106b may comprise a second material. As illustrated, the layers 104a, 104b, 106a 106b may alternate between the first material and the second material. The layers 104a, 104b, 106a 106b may have specific thickness so as to cause optical interference of incident light 110 that is reflected from each of the layers that results in either constructive or destructive interference and the desired output. In some designs, filter 100 is designed for a specific wavelength and the layers 104a, 104b, 106a 106b may have specific thickness, for example, such as a quarter of the wavelength. The layers 104a, 104b, 106a 106b, however, are configured to provide optical interference that results in high or low transmission (or reflectivity) for different wavelengths. In this manner, a desired spectral responsivity maybe designed for the filter 100. For example, the materials, thickness, and arrangement of the layers 104a, 104b, 106a 106b may be configured to provide a specific spectral characteristic such as a pass band. Accordingly, in some examples, the filter 100 comprises a band pass filter configured to selectively transmit or reflect a particular wavelength when light 110 is normally incident on the filter. FIG. 1B, for example, shows a schematic drawing of a spectral distribution 112 for a band pass filter. The band pass filter has a band pass region 114 where one or more wavelengths of light 110 incident on the filter 100 at normal incidence is transmitted therethrough. This band pass region 114 is show to be centered about the center wavelength of λ₀. In contrast, the spectral responsivity 112 of the band pass filter has spectral regions above and below the band pass region 114 that provides reduced transmission in comparison to the transmission of the band pass region for wavelengths above and below than the wavelengths of the band pass region.

Changing the orientation of the incident light 110 with respect to the filter 100 and the interference coating or alternatively changing the orientation of the filter with respect to the incident light can alter the spectral responsivity 112 of the filter. As illustrated in FIGS. 1C and 1D, for example, tilting the filter 100 with respect to the incident light 110 can shift the band pass region 114. In particular, this band pass region 100 shifts with tilt. The band pass region 114 for the tilted filter 100 shown in FIG. 1C, is centered about the center wavelength of λ₁, which is shifted with respect to the center wavelength of λ₀ of the un-tilted filter.

With further tilt, the spectral responsivity 112 continues to change. Further tilt of the filter 100 of FIGS. 1A and 1C is illustrated in FIGS. 1E and 1F. As shown in FIGS. 1E and 1F, for example, progressively tilting the filter 100 with respect to the incident light 110 can alter the band pass region 114 progressively more. In particular, this band pass region 114 is progressively shifted with tilt. The band pass region 114 for the tilted filter 100 shown in FIG. 1E, is centered about the center wavelength of λ₂, which is further shifted with respect to the center wavelength of λ₀ of the tilted filter of FIGS. 1A and 1s thus shown as shifted with respect to the center wavelength of λ₁ of the tilted filter of FIG. 1C.

Accordingly, progressively tilting or reorienting the filter 100 with respect to the incident light can change the transmission or reflection properties of the filter and thus changes the spectral distribution of light transmitted or reflected from the filter. For example, if white light is transmitted through the filter 100, a first wavelength band may be selectively pass through the filter and output therefrom. If that filter 100 is tilted slightly, a second wavelength band shifted slightly in wavelength with respect to the first wavelength band may be selectively passed through the filter and output therefrom. If that filter 100 is tilted slightly more, a third wavelength band shifted slightly more in wavelength with respect to the first wavelength band may be selectively passed through the filter and output therefrom.

The interference filter 100 need not be limited to the filter shown in FIGS. 1A-1F. For example, the interference filter 100 need not be a band pass filter but may for example be a long pass filter or a short pass filter. Additionally, although four layers 104a, 104b, 106a, 106b are shown, the interference coating may include a larger or smaller number of layers. Additionally, although two types of layers 104a, 104b, 106a, 106b each comprising a different material are shown, alternately, more than two different types of layers maybe be used. For example, the optical coating can comprise three different types of layers (e.g., a first group comprising a first layer comprising a first material disposed on a substrate, a second layer comprising a second material disposed on the first layer, and a third layer comprising a third material disposed on the second layer. This sequence of layers can be repeated again in a second group comprising another first layer comprising the first material, a second layer comprising the second material, and a third layer comprising the third material as stacked over the first group of layers. The different materials can have different indices of refraction. One may have a relatively high refractive index, one a medium refractive index, and one a relatively low refractive index. Although two groups of layers are described above, the coating may include more groups. Additionally, the groups may include more or less than three layers. Other variations are also possible.

FIG. 1G shows the filter 100 as tilted the opposite direction (counter clockwise) than the tilt (clockwise) shown in FIGS. 1D and 1F.

The substrate 102 on which the layers are may comprise glass, plastic, or other materials. In some embodiments the filter 100 is transmissive. Accordingly, the substrate 102 may be transmissive. In other cases, the filter 100 is reflective. Likewise, although transmission spectra 112 are shown in FIGS. 1B, 1D, and IF, these spectra may be reflective spectra and the output of the filter 100 may be reflected light that has a spectral distribution that can be modified in a gradual and continuous manner by tilting the filter.

Accordingly, actuators such as motors, piezos, etc. may be used to tilt and thereby reorient the filters in a controlled manner with respect to the incident light. Electrical signals may be applied to the actuators, e.g., motors, piezos, etc., to cause rotation. The electrical signal may be provided by electronics such as control electronics that controls the amount of tilt of the filter(s) and hence the amount of change of spectral responsivity of the filter and thus the spectral distribution of the light output (e.g., transmitted through or reflected from) the filter or filters. In certain embodiments, multiple filters (e.g., a high pass filter and a low pass filter) can be placed in a single light path. For example, the multiple filters can be stacked on a single surface, on opposite sides of a plate or element, and/or coated on a surface or element and embedded within an epoxy bond. The electronics may also control the light emitters (e.g., LEDs, laser diodes), for example, the amount of light output by the light emitter. Such electronics, may for example, control the amount of electrical power that drives the emitters.

Accordingly, illumination systems may include light sources such as LEDs that output light that is directed along one or more paths. Tunable filters such as described above may be included in the one or more paths to alter the spectrum of the light from the light sources that propagate along the one or more paths. These paths maybe brought together to provide an aggregate beam having the desired spectral distribution.

In some examples, therefore, thin film coatings, such as interference coatings, applied to one or more plane parallel plates or other substrate are placed in the paths of corresponding beams (e.g., collimated beams) and can be adjustably tilted to vary the color or waveband distribution.

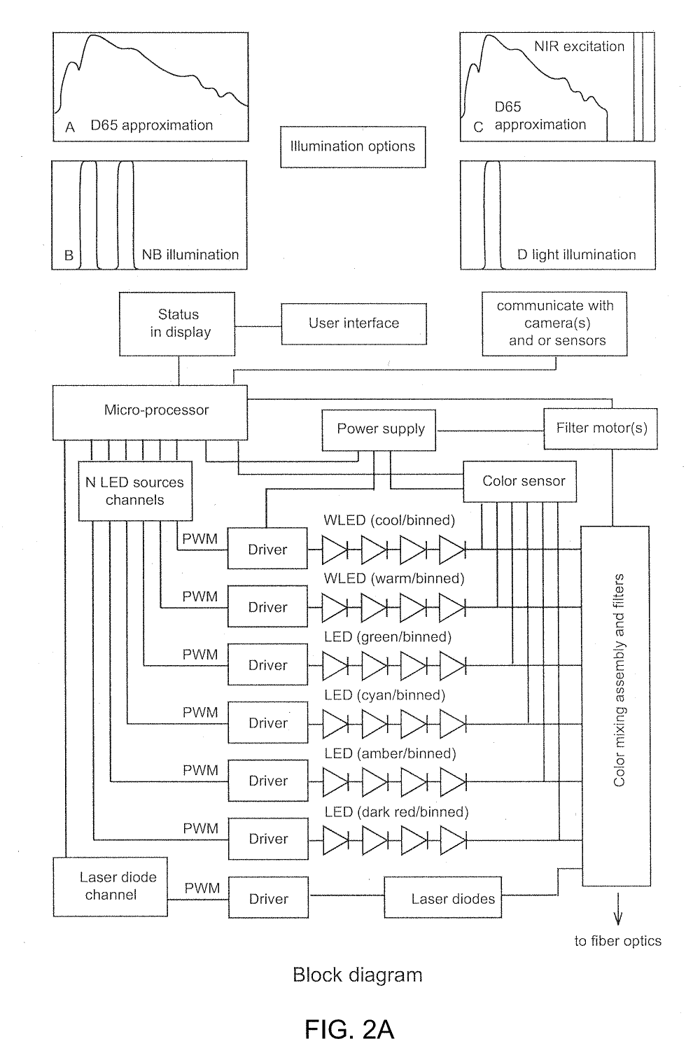

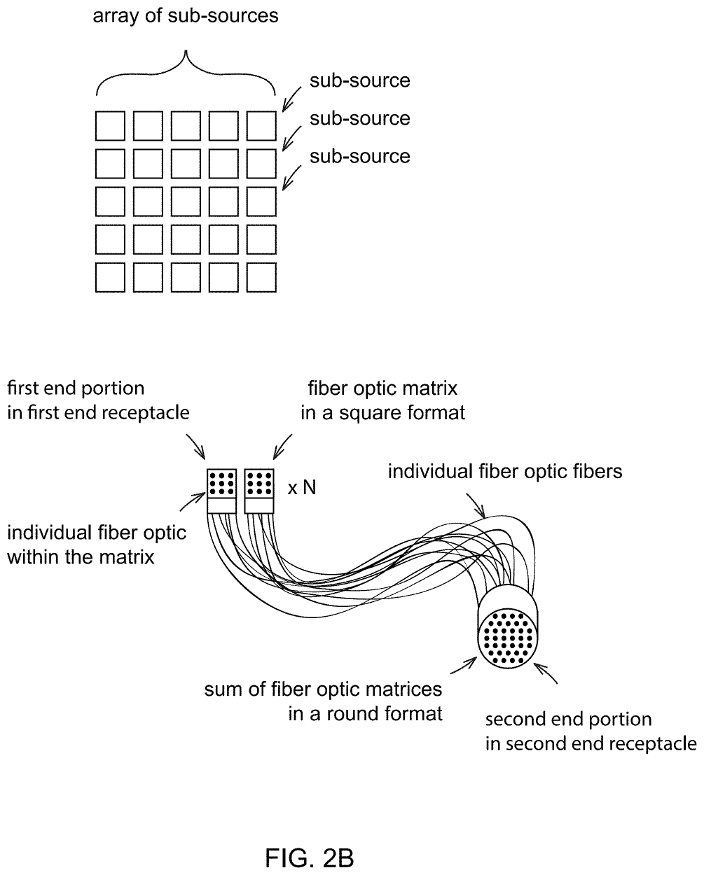

FIG. 1H schematically illustrates an example color mixing assembly 140 comprising a plurality of filters 100 in accordance with certain embodiments described herein. The example color mixing assembly 140 may be used in the example illumination device schematically illustrated in FIG. 2A and described more fully below. The color mixing assembly 140 comprises a first waveguide 150a (e.g., glass or plastic; mixing rod; light guide or waveguide) having a first output face 152a, a first lens assembly 160a, the plurality of filters 100, a second lens assembly 160b, and a second waveguide 150b (e.g., glass or plastic; mixing rod; light guide or waveguide) having a second output face 152b and an input face 154b. The first output face 152a is in optical communication with the first lens assembly 160a. The first lens assembly 160a is in optical communication with the plurality of filters 100, the plurality of filters 100 is in optical communication with the second lens assembly 160b, and the second lens assembly 160b is in optical communication with the input face 154b of the second waveguide 150b. The plurality of filters 100 is configured to direct the light emitted by the plurality of filters 100 to the second lens assembly 160b. The second lens assembly 160b is configured to focus the light and to couple the light to the second waveguide 150b via the input face 154b of the second waveguide 150b. The second output face 152b of the second waveguide 150b is configured to direct the light to the fiber optic cables (not shown). For example, the second output face 152b can be in close proximity to the fiber optic cables. Light from the optical emitters is directed to the first waveguide 150a, propagates through the first output face 152a, through the first lens assembly 160, the plurality of filters 100, the second lens assembly 160b, and the input face 154b of the second waveguide 150b, with the resultant light emitted from the second output face 152b of the second waveguide 150b.

The first output face 152a of the first waveguide 150a is configured to emit light directed towards the first lens assembly 160a which is configured to substantially collimate the light emitted from the first output face 152a and to direct the collimated light along the optical path 170 through the plurality of filters 100. Each filter 100 of the plurality of filters 100 comprises at least one interference plate 102 having at least one dichroic coating applied to at least one face of the plate 102, and the plate 102 is configured to be controllably rotated (e.g., tilted) in at least one direction. The angular and/or rotational differences of the plates 102 are configured to be controllably adjusted such that the filters 100 controllably interact with the light received from the first lens assembly 160a to modify (e.g., shift; compress) the spectral distribution of the light in an advantageous manner. For example, referring to FIGS. 1C and 1D, the spectral distribution of the light can be shifted and compressed from λ0 to λ1.

The plurality of filters 100 has a center axis 170 along which the light is propagating from the first lens assembly 160a to the second lens assembly 160b in a region between the first lens assembly 160a and the second lens assembly 160b and the plates 102 of the plurality of filters 100 are located along the center axis 170. The various views of the color mixing assembly 140 in FIG. 1H show various example degrees of freedom in which the plates 102 may be configured to be controllably rotated (e.g., tilted). In view (i) of FIG. 1H, the two plates 102 are both generally perpendicular to the center axis 170. In view (ii) of FIG. 1H, one plate 102 is configured to be rotated about an axis perpendicular to the center axis 170 and parallel to the plate 102, and the other plate 102 is configured to be rotated about an axis parallel to the center axis 170. In view (iii) of FIG. 1H, one plate 102 is configured to be rotated about an axis perpendicular to the center axis 170 and parallel to the plate 102, and the other plate 102 is configured to be rotated about an axis perpendicular to the center axis 170 and parallel to the plate 102, with the two rotational axes parallel to one another. In view (iv) of FIG. 1H, one plate 102 is configured to be rotated about an axis perpendicular to the center axis 170 and parallel to the plate 102, and the other plate 102 is configured to be rotated about an axis perpendicular to the center axis 170 and parallel to the plate 102, with the two rotational axes perpendicular to one another.

In various embodiments, the plurality of filters 100 can comprise one or more dichroic filters. In some embodiments, the plurality of filters 100 can comprise one or more polarization components. Rotating or tilting one or more components of the plurality of filters 100 (e.g., the plate 102) about the different rotational axes can induce a spectral change in the filter output. For example, rotating or tilting one or more components of the plurality of filters 100 (e.g., the plate 102) can attenuate, reduce or extinguish a portion of the signal output from the plurality of filters 100. As another example, rotating or tilting one or more components of the plurality of filters 100 (e.g., the plate 102) can shift the spectrum of the signal output from the plurality of filters 100.

In various embodiments, the plurality of filters 100 comprising one or more dichroic filters can be sensitive to polarization of light output from the one or more light sources. In such embodiments, rotating or tilting (e.g., rotating azimuthally) the one or more components of the plurality of filters 100 (e.g., the plate 102) can change the spectral composition of the light output from the plurality of filters 100. For example, if in a particular orientation of the various components of the plurality of filters 100, the output from the plurality of filters 100 can comprise a first amount of light in a first wavelength and a second amount of light in a second wavelength. When one or more components of the plurality of filters 100 is rotated or tilted, the output from the plurality of filters 100 can include different amounts of light in the first and the second wavelengths. This effect may result because the filters may be polarization dependent in some cases. The resultant change in transmission of different wavelengths through the filter(s) can thus modify the spectral composition of the output of the filters. By increasing and/or decreasing the intensity of different wavelengths output by one or more such filters, for example, the overall shape of the spectral distribution can be altered. For example, the magnitude of certain spectral wavelengths can be reduced compared to other wavelengths. Accordingly, various optical emitters can be tailored to emit light having a desired spectral characteristic and/or intensity characteristic which when combined with rotation or tilt of the one or more components of the plurality of filters 100 can provide light with desired illumination characteristics (e.g., spectral characteristic and/or intensity characteristic).

FIG. 1I schematically illustrates another example color mixing assembly 140 comprising a plurality of filters 100 in accordance with certain embodiments described herein. The color mixing assembly 140 shown in FIG. 1I does not include airspaces or lenses through which light is transmitted while propagating through the color mixing assembly 140. As compared to the example color mixing assembly 140 of FIG. 1H, the example color mixing assembly 140 of FIG. 1I replaces the lens assemblies 160a, 160b with first and second tapers 180a, 180b. The first taper 180a comprises an input end 182a in optical communication with the output face 152a of the first waveguide 150a and an output end 184a in optical communication with the plurality of filters 100. The size of the input end 182a (e.g., diameter; width; area) is smaller than the size of the output end 184a (e.g., diameter; width; area) such that a numerical aperture of the input end 182a is smaller than a numerical aperture of the output end 184a. The second taper 180b comprises an input end 182b in optical communication with the plurality of filters 100 and an output end 184b in optical communication with the input face 154b of the second waveguide 150b. The size of the input end 182b (e.g., diameter; width; area) is larger than the size of the output end 184b (e.g., diameter; width; area) such that a numerical aperture of the input end 182b is larger than a numerical aperture of the output end 184b. Thus, the tapers 180a, 180b of FIG. 1I serve a similar function as do the lens assemblies 160a, 160b of FIG. 1H.

In certain embodiments, the reduced numerical aperture, or angular output, of the color mixing assembly 140 (e.g., whether by lens assemblies 160a, 160b of FIG. 1H or by tapers 180a, 180b of FIG. 1I) can be used to produce a more collimated flux profile permitting the plurality of filters 100 to be more efficient in modifying (e.g., shifting; compressing; blocking; passing) portions of the flux energy from the optical emitters. FIG. 1I schematically shows a light ray entering the waveguide 150a at an angle and traversing the length of the waveguide 150a until transitioning into the taper 180a, where upon the light ray reflects off a side wall of the taper 180a at a new angle associated with the taper 180a. The sum of the energy of the light ray remains substantially unchanged (e.g., except for small side wall losses and coupling losses).

The flux energy exiting the plurality of filters 100 is coupled to the waveguide 150b by the taper 180b. As shown in FIG. 1I, the ray paths of the light propagating through the plurality of filters 100 can be steeply angled relative to the center axis 170 of the plurality of filters 100.

Each of the two plates 102 of the plurality of filters 100 can be configured to be controllably rotated (e.g., tilted) with respect to one another, thereby permitting modification (e.g., shifting; compressing; blocking; passing) portions of the flux energy propagating through the color mixing assembly 140. For example, each of the two plates 102 can be configured to be controllably rotated (e.g., tilted) about one or more axes (e.g., the center axis 170 of the plurality of filters 100; an axis perpendicular to the center axis 170 and parallel to the plate 102; an axis perpendicular to the center axis 170).

FIG. 1J schematically illustrates example portions of example color mixing assemblies 140 in accordance with certain embodiments described herein. While the waveguides 150, lens assemblies 160, tapers 180, and plates 102 of certain embodiments can be cylindrical and circularly symmetric, as in view (i) of FIG. 1J, in certain other embodiments, one or more of the waveguides 150, lens assemblies 160, tapers 180, and plates 102 can include one or more flat sides. Certain such embodiments can advantageously skew the light rays, mix the flux energy, and/or integrate the flux energy propagating through the color mixing assembly 140 to generate a more even light output. For example, the four-sided waveguide 150 shown in view (ii) of FIG. 1J can provide a more even light output than can the circularly symmetric waveguide 150 shown in view (i) of FIG. 1J, and the six-sided waveguide 150 (e.g., hex rods) shown in view (iii) of FIG. 1J can provide a more even light output than can the four-sided waveguide 150. In certain embodiments, the interfaces between the larger ends of the tapers 180 with faceted sides can suffer more losses than do interfaces with the circularly symmetric tapers 180, particularly in embodiments in which the plates 102 have circular diameters. In certain embodiments, an advantageous compromise can be achieved using a six-sided waveguide 150 and a six-sided taper 180. In certain embodiments, a taper 180 having facets on one end and circularly symmetric on the other end can be used. In certain embodiments, the tapers 180 and waveguides 150 (e.g., hex rods) of FIGS. 1I and 1J can rotate axially with respect on one another and with respect to the sections containing the plates 102. To facilitate optical coupling between the rotating surfaces, a matching index fluid may be disposed between the tapers 180 and the sections containing the plates 102. In some configurations, for example, the light can propagate between the taper and the index matching fluid and between the index matching fluid and waveguide thereby reducing Fresnel reflection from the surface of the taper and the surface of the waveguide as a result of the presence of index matching fluid. The index matching fluid, may for example have an index of refraction greater than 1.0. The index matching fluid may have an index close to the index of the tapers and the waveguides.

The one or more plates (filters) can be coupled to an electrical controller configured to simultaneously change the power provided to some or all of the LEDs in groups or together as the plates are tilted. The light source can also communicate with a control device which may display spectral power distribution to a visualization display. Certain such embodiments advantageously provide high power densities using arrays or assortments of low-cost, high-output LED dies. In certain embodiments, one or more LEDs can be placed in direct contact with one or more waveguides and can direct their flux energy in the direction of the long axis of the waveguide, with the flux energy captured in the waveguide by total internal reflection.

In certain designs, for example, the illumination system or light source may contain a communication bus, which communicates with one or more cameras. The color responses can vary greatly between cameras, and in certain cases, an input profile can be provided specifically for the camera. Certain configurations can provide an illumination system that tailors its output for different cameras used in switching the resultant visualization. The illumination system may, for example, adjust the spectral or color waveband distribution depending on which camera is being used to generate the image being viewed by the user.

In certain illumination systems, the variable spectral output generated using the tilted plates can be advantageously used with corresponding filters in the one or more cameras. For example, autofluorescence and exogenous agents utilize intense excitation sources that can obscure the emission of dyes and agents used in many studies. In such circumstances, the camera can include blocking filters to be used in conjunction with the light source, and the variable output can be adjusted accordingly. For example, the cameras can include filters or detectors that are configured to block light below 700 nm, such that autofluorescence largely disappears in images with wavelengths above 700 nm, so fluorescence imaging in the infrared reduces background “noise” caused by tissue autofluorescence.

Furthermore, color rendering in normal visualization and false-color and pseudo-color rendering can benefit from variable filtering with tilted plates in some instances. For example, the color rendering in normal visualization may be more medically useful to the physician if the color temperature of the light is modified, and/or if the illumination or brightness level can be varied or modified (e.g., by excluding, enhancing, or otherwise modifying one or more portions of the waveband) by the introduction of one or more variable filters used alone or in combination with one another (e.g., blocking or passing filters). In certain embodiments, the illumination system is configured to be used with a visualization system that incorporates false color and/or pseudo-color images.

As used herein, “false color” refers to a group of color rendering methods used to display images in color which were recorded in the visible or non-visible parts of the electromagnetic spectrum, and a false-color image is an image that depicts an object in colors that differ from those a true-color image would show. A false-color image can be created using solely the visual spectrum (e.g., to accentuate color differences), and/or using data from electromagnetic radiation outside the visual spectrum (e.g., infrared, ultraviolet, X-ray), with the choice of spectral bands governed by the physical properties of the object under investigation. In addition, variants of false color (e.g., pseudo-color, density slicing, and choropleths) can be used for information visualization of either data gathered by a single grayscale channel or data not depicting parts of the electromagnetic spectrum (e.g., elevation in relief maps or tissue types in magnetic resonance imaging). In contrast to a true-color image, a false-color image sacrifices natural color rendition in order to ease the detection of features that are not readily discernible otherwise (e.g., the use of near infrared for detecting emission from an exogeneous dye; imaging tissue features hidden below the surface which are visible in the near infrared, but not visible in visible light, such as in a range of 400 nm-700 nm).

In certain designs, the illumination device can include one or more tilting planes and a mix of phosphor-converted LEDs (e.g., white LEDs; blue or purple LEDs coated with a phosphor to reemit over a broader and longer wave band range), multi-colored LEDs (e.g., a plurality of LEDs of two or more different colors), and/or one or more other excitation sources (e.g., near-IR). As an example, various white LEDs can be blue or purple with overlaid phosphors to “Stokes shift” the output to longer wavelengths. There may be a tension between wanting output versus spectral quality. With less phosphor, the resulting bluish light can have more power. With more phosphor, there may be a yellow or warmer shift at the expense of output power. Adding one or more tilting plates with a dichroic coating(s) can remove some of the excess blue. In addition, adding sources for extended red can help produce a more useful illuminator for surgery. As another example, in photodynamic therapy applications, which utilize illumination in the UV and blue regions (e.g., soret band range), suitable excitation sources can be added to the device and controlled as other modes. In some examples, 5ALA, (also dALA, δ-ALA, or 5-aminolevulinic acid), an endogenous non-proteinogenic amino acid, which can be useful in cancer identification, e.g., in particular malignant gliomas in neurosurgery, can utilize similarly configured excitation sources and blocking filters. Additionally, in other examples, near-IR excitation sources can be used with visual illumination or narrow-band imaging (NBI).

FIG. 2A is a block diagram schematically illustrating an example light source or illumination device. The example light source or illumination device includes a plurality of sub-source channels or sub-assemblies configured to generate light, and a controller subsystem comprising a micro-processor and/or other electronics, a color sensor, a color mixing assembly and one or more filters, and one or more filter motors. The micro-processor and/or electronics is operatively coupled to the plurality of sub-sources or sub-assemblies configured to generate light, the color sensor, and the one or more filter motors. The output of the color mixing assembly and one or more filters may be operatively coupled to fiber optics.

Certain embodiments described herein can utilize one or more color sensors having a color scheme division of the spectrum. Examples of color scheme divisions include a red-green-blue (RGB) color scheme division, a cyan-magenta-yellow-black (CMYK) color scheme division, a hue-saturation-value (HSV) color scheme division, or another color scheme division of the useful spectrum. Each color sensor can have one or more portions that are responsive to flux from corresponding portions of the spectrum (e.g., from green or cyan), or one or more portions that are responsive to one or more corresponding colored LEDs or groupings of LEDs. The color sensor can be configured to receive a portion of the total flux from the at least one optical emitter. For example, a portion of the mixing assembly (e.g., waveguide; mixing rod) can use total internal reflection to move flux in a direction from the light source to the output and a portion of the surface of the mixing assembly can comprise a dichroic coating which passes one or more wavelengths of interest to a color sensor. This portion of the flux can be coupled directly to the color sensor (e.g., by contact; via a fiber optic assembly; via a waveguide or mixing rod) to allow the color sensor to sample the flux for its spectral properties. In certain embodiments, the output spectrum of the light source can be managed by sampling the subdivisions of the flux in the waveguide (e.g., mixing rod) and by adjusting the power supplied to one or more of the LEDs (e.g., via the microprocessor and user interface), for example, to adjust the spectral characteristics of the light based on the samples obtained using the wavelength specific or color sensors.

In the design shown in FIG. 2A, each sub-source of the plurality of sub-sources comprises at least one optical emitter (e.g., an LED or laser diode) and at least one pulse-width modulation (PWM) circuit operatively coupled to the at least one optical emitter and configured to respond to control signals from the micro-processor or other electronics to drive the at least one optical emitter that is responsive to signals from the PWM circuit. For example, the output of the LEDs (e.g., in lumens) can be controlled by a PWM circuit generating digital signals (e.g., having a number of bits, such as 4 bits, 8 bits, 10 bits, with larger numbers of bits providing finer gradations of intensity levels) and a serial interface configured to transmit the digital signals to the corresponding optical emitters.

FIG. 2B schematically illustrates a portion of an example illumination device in accordance with certain embodiments described herein. The example illumination device shown in FIG. 2B comprises at least one sub-source (e.g., one or more pluralities of sub-sources, one or more arrays of sub-sources; one or more sub-source matrices) comprising one light emitter or a plurality of light emitters (e.g., LEDs) configured to produce light flux. The example illumination device further comprises a plurality of optical fibers (e.g., a fiber matrix). The different (e.g. each) optical fibers of the plurality of optical fibers may comprise a first end portion configured to receive the light flux from a corresponding light emitter or from a plurality of light emitters and a second end portion configured to emit the received light flux. The one or more light emitters are arranged in a first pattern. The first end portions may be arranged in the first pattern. and/or the second end portions may be arranged in a second pattern different from the first pattern.

For example, the at least one sub-source schematically illustrated in FIG. 2B comprises an array of square sub-sources. The light emitters of the array of square sub-sources are arranged in a first pattern (e.g., the array of sub-sources arranged in a rectilinear, square, or rectangular sub-pattern, and the light emitters of different sub-sources (e.g. each sub-source) also arranged in a rectilinear, square, or rectangular sub-pattern). The plurality of first end portions can be arranged in the same first pattern. The plurality of second end portions can be arranged in a different second pattern (e.g., a circular pattern; a second rectilinear pattern different from the first rectilinear pattern).

The plurality of optical fibers can be mechanically coupled together (e.g., contained in a matrix of adhesive such as epoxy or fused by temperature) in at least one first end assembly (e.g., at least one input receptacle) containing the first end portions and at least one second end assembly (e.g., at least one output receptacle) containing the second end portions. For example, as schematically illustrated by FIG. 2B, the at least one first end assembly can comprise a fiber optic matrix in a non-round (e.g., square; rectangular) format and the at least one second end assembly can comprise a sum of fiber optic matrices in a non-square format (e.g., round format). Likewise, the at least one first end assembly can comprise a fiber optic matrix in a non-round format and the at least one second end assembly can comprise a sum of fiber optic matrices in a different non-round format. The number of first end assemblies can be greater than the number of second end assemblies. For example, the number of first end assemblies can be 4, 9, 16, 25, 36, 49, or N² (e.g., each having a square format and configured to be in optical communication with a 2×2, 3×3, 4×4, 5×5, 6×6, 7×7, or N×N square array of square LEDs, respectively) and the number of second end assemblies can be 1, 2, 4, 6, 8, or less than N² (e.g., each having a circular format). Other examples can utilize non-square numbers (e.g., M×N, with M not equal to N, arranged in a rectangular format or other formats).

In certain embodiments, a first set of the first end portions can be in optical communication with the light emitters of a first sub-source and a second set of the first end portions can be in optical communication with the light emitters of one or more other sub-sources (e.g., a second sub-source; a third sub-source; N sub-sources). For example, a first set of the first end portions in a square end assembly and a second set of the first end portions in a different square end assembly can receive the light flux from the respective light emitters, and the second end portions of these optical fibers can be gathered into one or more circular end assemblies (e.g., to facilitate mixing of the received light flux from the respective light emitters). Such mixing may be accomplished, for example, by having the relative locations and/or order of different fibers in the first set of the first end portions be different from the relative locations and/or order of those same fibers in the second set of the first end portions In certain such embodiments, combining the light flux from various sub-sources (e.g., having differing color spectrums, power spectral densities, etc.) at the first end portions of the first end assemblies to be emitted from the second end portions of the second end assemblies which may have different arrangement and/or order, advantageously increases randomization of the flux output.

The plurality of optical fibers can be flexible (e.g., configured to be moved such that the second end portions are positioned at various selected locations relative to the first end portions). For example, the flexible plurality of optical fibers can be configured to allow the first end portions to be coupled to (e.g., adjacent to) the plurality of sub-sources (e.g., such that each first end portion is in optical communication with a corresponding one or more of the light emitters of a sub-source) and the second end portions to be coupled to (e.g., adjacent to) a filter assembly, lens assembly, mixer assembly, or an assembly combining any combination of filters, lenses and/or mixers (e.g., such that each second end portion is in optical communication with a corresponding portion of the filter or lens assembly, as described herein). For example, the individual optical fibers making up the fiber matrix can each have an outer diameter or lateral dimension selected to provide a desired flexibility (e.g., an outer diameter or lateral dimension of 20 microns, 30 microns, 50 microns, 70 microns, 80 microns, or in any range formed by any of these values). In certain other embodiments, the plurality of optical fibers can be fixed and the at least one sub-source can be configured to move, such that light emitters of a selected one or more sub-sources of the at least one sub-source are placed in optical communication with at least one selected set of optical fibers.

In certain embodiments, the sum of the areas of the sub-sources (e.g., areas of the input square formats) is substantially equal to the area of the output (e.g., circular output format) that is in optical communication with the sub-sources. In certain other embodiments, the first end portions of the individual optical fibers can be tapered (e.g., such that the numerical aperture of the fiber matrix is greater than or less than the input face of the fiber optic matrix).

In some implementations, the plurality of optical fibers could be heated and drawn producing a taper and/or tapers that could be combined together. In some implementations, the tapers could be different from one another and combined. The numerical aperture (NA) of a fiber may change as the area of the fiber changes. According, by tapering a fiber and producing a smaller diameter or cross-sectional area, the numerical aperture at that end can be caused to be increased. Decreasing the area of the second end portion, for example, may increase the numerical aperture of the second end portion and thus may increase the output angle or divergence angle of light exiting the optical fiber at the second end portion. Alternatively, one could orient the taper the other way. The first end portion of the fiber may be tapered to produce a smaller diameter or cross-sectional area at the first end compared to the first end portion. Consequently, the numerical aperture at the first end portion can be caused to be increased relative to the second end portion. The first end portion can thus accept more light from an LED by using the smaller end at the source and using the larger end where light may be output to focusing, collimating and/or mixing optics. Tapering and reducing the cross-section fiber at the input (e.g., reducing the cross-sectional area at the first end portion compared to the second end portion) can be used to capture more illumination or at the output (e.g., reducing the cross-sectional area at the second end portion compared to the first end portion) can be used to match the acceptance angle of an optical system, thereby improving efficiency.

Having different shaped formats for the first and second end assemblies can be useful in some instances to address the shape of structures in which emitters are packages. Emitters such as light emitting diodes (LEDs) may be included in square shaped LED sources such as LED arrays. As disclosed herein, a number of small LEDs can be optically coupled to a plurality of fiber matrices having square shape and then transmitted some distance from the emitters to a surgical device or transmitted internally within a surgical device. Light from the emitters may propagate through the fibers to one or more of the second end assemblies, which may be circular in some implementations as discussed herein. In some implementations, the number of second end assemblies including the second end portions is less than the number first end portions of fiber, which may be disposed proximal to the emitters. For example, one could have a 5×5 array of LED's coupled with one or more square fiber matrices and then gather these 25 fibers together into 1, 2, 3, 4, etc. circular second assemblies.

Systems, devices, and apparatus disclosed herein may be used to distribute light from one or more first end assemblies into a plurality of second end assemblies such as circular assemblies. Such systems, devices, and apparatus may be configured to directing light into focusing optics having a smaller diameter than the aggregate area of the emitters from which light originates. For example, the coupling fibers can permit coupling the optical power of a large LED or LED array into a plurality (e.g., 2, 3, 4, etc.) of focusing optic assemblies that individually have smaller areas than the larger LED or LED array. Similarly, the coupling fibers can permit coupling the optical power of one or more first end assemblies into a plurality (e.g., 2, 3, 4, etc.) of second end assemblies individually having smaller areas than the total cross-sectional area of the one or more first end assemblies.

In certain embodiments, sub-sources (e.g., arrays of sub-sources) can be configured to be moved individually or as a group to select at least one sub-source (e.g., at least one array of sub-sources) to be in optical communication with the plurality of first end portions in the at least one first end receptacle. For example, the sub-sources can be mounted on a support configured to move (e.g., rotate about a hub as the center of rotation; slide along a track), with the support configured to be positioned to place at least one sub-source (e.g., an array of sub-sources) in optical communication with the plurality of first end portions. Different sub-sources on the support can have characteristics that are different from the characteristics of the other sub-sources (e.g., one array of sub-sources can comprise light emitters with phosphors to emit white light; another array of sub-sources can comprise light emitters having a different color spectrum, power spectral density, etc.). These sub-sources (e.g., arrays of sub-sources) can be at different positions on the support (e.g., four arrays of sub-sources at the “12 o'clock,” “3 o'clock,” “6 o'clock,” and “9 o'clock” positions of a rotating support), and the support can be positioned to place the array(s) having the desired characteristics (e.g., color spectrum) in optical communication with the plurality of first end portions. In certain other embodiments, the plurality of optical fibers can be fixed and the sub-sources (e.g., arrays of sub-sources) can be configured to be moved as a group to select at least one sub-source (e.g., at least one array of sub-sources) to be in optical communication with the plurality of first end portions in the at least one first end receptacle.

In certain embodiments, the portion of the illumination device advantageously allows the packing fraction of the optical fibers to be dimensionally less than the spacing of sub-sources. In some cases, the spacing of illumination devises or emitters can be impacted by thermal management considerations.

In some implementations, the light emitters are generally small and can be moved while the fiber optic matrix remains stationary and provides the light flux to additional optical systems or sub-systems (e.g., collimating assembly, mixing assembly, or focusing lens assembly or combinations thereof). In certain embodiments, the portion of the illumination device advantageously provides light flux from a square or rectangular array of sub-sources to optical systems or sub-systems that have different geometries (e.g., systems having a circular aperture or cross-section or field of view and/or that possibly see or transmit an image circle or light bundle).

The illumination device may comprise an illuminator that may be mounted on a stand or attached to different fixtures such as supports. The illumination device can be brought near the patient possibly to supplement overhead surgical lighting (e.g., used by the physician sans optical devices or with non-illuminated magnification devices, such as loupes, or to supplement other medical imaging modalities such as endoscopes, exoscopes, or cameras providing surgical microscope views). In addition or alternatively, the illumination device may be integrated with other medical imaging devices such as cameras providing surgical microscope views, etc.)

A range of advantages may be obtained using designs and configurations disclosed herein. For example, designs disclosed herein may facilitate mixing of light from LEDs that having different color that are selectively activated to provide a desired spectrum. Certain color lights may be added using selectively activated color emitters (e.g., LEDs) to supplement an otherwise discontinuous spectrum of white phosphor LEDs.

Additionally, a plurality of smaller emitters (e.g., LEDs) with gaps between them may have less thermal load than a single larger emitter (e.g., LED). Systems, devices, and apparatus disclosed herein may be used to combine light from such a plurality of smaller emitters. Systems, devices, and apparatus disclosed herein may be configured to direct light into smaller diameter focusing optics. Coupling fibers for example can permit coupling the power of a large LED into four smaller focusing optics.

A wide range of variations in the illumination device are possible. For example, the shapes and arrangements of the array of sub-sources as well as fiber optic matrix may be different. For example, shapes different from those disclosed for each of the components are possible. Likewise shapes other than square, rectangular, circular are possible. Additionally, although movement of the emitters has been described above, in some implementations the fiber may be moved. For example, the first end portions (e.g., the first end assembly) can be moved with respect to the emitters, sub-sources, and or second end portion in the second end assembly. Also, one or both of the first and second end assemblies can merely comprise fused fibers and does not include any extra components attached to the fibers. Additionally, any systems, devices, components, and/or features described herein can be combined with any other systems, devices, components, and/or features described herein. For example, any systems, devices, components, and/or features described in connection with FIG. 2B can be combined with any other systems, devices, components, and/or features described elsewhere herein, including with respect to any of the other Figures.

In certain embodiments, the at least one PWM circuit can be used to control the optical emitters to provide flicker-free illumination. Time-variant light artifacts (TLAs), commonly called fluctuations or flicker, are noticeable to most humans at frequencies below 70 Hz. Some people are sensitive in their central vision region to TLAs with frequencies up to 100 Hz, while using peripheral vision, TLAs can be perceived with frequencies up to 200 Hz. For wide field of view imaging systems, it is desirable to manage the illumination to improve (e.g., optimize) “time variant light quality,” especially since such wide field of view imaging systems engage the peripheral vision of the user. By comparison, endoscopic images viewed through ocular systems have narrow apparent fields of view (e.g., 10 to 30 degrees). Viewing a medical image on a monitoring screen in 2D or 3D (e.g., attached to an arm and surgical stand) may well engage fields of view of 30 to 45 degrees. A surgical microscope or electronic near eye display (e.g., fixed on an arm or worn) can have an apparent field of view of 60 to 90 degrees, and immersive displays (e.g., head mounted or fixed on an arm) can engage nearly the entire periphery of a user's vision system. Thus, in certain embodiments described herein the illumination system utilizes the at least one PWM circuit to control the generated light to conform to user requirements for perceptually flicker free illumination (e.g., substantially above 200 Hz). For example, the at least one PWM circuit can be configured to control the generated light to direct light onto the optical emitters at a pulse rate sufficiently fast to avoid detection of flicker by the user, in one or both of the user's central viewing region and the user's peripheral viewing region.

In certain embodiments, the at least one PWM circuit can be used to compensate for differences between the color characteristics of different medical cameras. For example, single-chip medical cameras and three-chip medical cameras have different color characteristics due to their color separation filtering. In some cases, for example, single-chip cameras can use a Bayer filter arrangement with different color filters over different pixels in a repeating pattern, while three-chip cameras can use three dichroic filters arranged to produce three different color channels (e.g., a red, green, or blue channel) each with its own sensor array. The pixels in these sensor arrays used in a three-chip camera have their respective color channels that are sensitive primarily in different respective portion of the spectrum (e.g., red, green, blue, etc.) though there can be spectral or wavelength overlap among the different sensor arrays. The output in color space of these two families of cameras can produce slightly different responses. Additionally, medical cameras from different manufacturers may use different sensor arrays which can add to the differences. Using the at least one PWM circuit to compensate for differences between different sensor arrays having different spectral characteristics or spectral responsivities. For example, using the at least one PWM circuit to compensate for differences between the color characteristics of these different medical cameras can advantageously allow better color matching between single-chip sensors and three-chip sensors and between the products from different camera manufacturers. The modulation circuitry can modulate the at least one light source (e.g. at least one LED) differently for different sensor arrays or cameras.

Various optical emitters can be configured to output illumination having one or more characteristics (e.g., intensity, wavelength, etc.). For example, some optical emitters can be configured to output optical power that has a spectral distribution similar to a CIE standard Illuminant D65. The characteristics of the light output from an optical emitter can be configured to match the detection capabilities of the various cameras that are configured to view the illumination output from the optical emitter. The optical emitter can be configured to adjust the characteristics of the light output (e.g., using the modulation circuitry) to more closely match the detection capabilities of the camera/sensor viewing the illumination output from the optical emitter. For example, the optical emitter can comprise a look up table of settings that includes the illumination characteristics that more closely match the various cameras/sensors that can view illumination output from the optical emitter. Various cameras/sensors can identify themselves over a communication link or bus, and the optical emitter can adjust the characteristics of the light output to more closely match the detection capabilities of the identified cameras/sensors. This feature can be advantageous when one or more cameras/sensors are switched on or off or are switched from being used to present images to not being used to present images or vice versa.

In certain embodiments, the at least one PWM circuit can be used to control the optical emitters to conform to various regulations regarding their use. For example, illumination is a time-based quantity, and the production of heat in tissue caused by the illumination is also a time-based quantity. For another example, the optical emitters can comprise one or more laser diodes (e.g., for excitation of an exogeneous dye), in which case the illumination source may potentially be subject to compliance with various regulatory requirements.

As shown in FIG. 2A, the example illumination device comprises N LEDs sub-source channels and a laser diode sub-source channel. One or more LED sub-source channels and in some cases each LED sub-source channel may comprise a PWM circuit and a plurality of corresponding LEDs operatively coupled to the corresponding PWM circuit, and the laser diode sub-source channel comprises a PWM circuit and one or more laser diodes operatively coupled to the corresponding PWM circuit. The PWM circuits are operatively coupled to the power supply. For example, as shown in FIG. 2A, six LED sub-source channels each have a corresponding PWM circuit and a corresponding set of LEDs, with each LED sub-source channel generating light in a corresponding spectrum: WLED (cool/binned), WLED (warm/binned), LED (green/binned), LED (cyan/binned), LED (amber/binned), and LED (dark red/binned). As used herein, “binned” refers to a collection of optical emitters (e.g., LEDs) with one or more similar properties and that are grouped together. The bins or groups can be defined by one or more properties including but not limited to, intensity distribution plots, lumen output, color temperature, and voltage. For example, a collection of LEDs with similar lumen output, but slightly different color temperatures within the green range can be binned together in a LED sub-source channel. Using bins can be advantageous by widening the peak distribution of the spectral peak of the light generated by the LED sub-source channel (e.g., for LEDs having individual colors, such as red, green, blue, amber, cyan, etc., and for white light LEDs). Each of the N LED sub-source channels also comprises one or more corresponding color sensors which are operatively coupled to the power supply and to the output of the corresponding optical emitters of the channel.

Optical signals outputted from the optical emitters are transmitted to a color mixing assembly and filters configured to generate light having a corresponding spectrum and to provide this light to the fiber optics.

In the example light source shown in FIG. 2A, in a seventh sub-source channel, a plurality of laser diodes are controlled by digital signals received from a corresponding PWM circuit, and optical signals outputted from the laser diodes are transmitted to the color mixing assembly and filters. Other numbers and combinations of sub-sources/sub-source channels and emitters are possible. Additionally, the illumination device may include additional components or may exclude one or more of the components shown and/or the arrangement of components may be different. Other features may be varied as well.

The light from the outputs of the plurality of sub-sources is directed to the one or more filters inputted into the color mixing assembly. For example, the plurality of sub-sources can generate corresponding light beams, the light beams can be transmitted through corresponding filters of the one or more filters to the color mixing assembly. In some cases, the color mixing assembly combines the light beams into a single composite beam. In some cases, the color mixing assembly can comprise at least one collimator configured to collimate the light beams to a single composite light beam. In certain designs, the light source further comprises an optical focusing assembly to converge the composite light beam and to transmit the composite light beam to a receiving fiber optic conduit or cable.

The color sensor can be operatively coupled to the outputs of the plurality of sub-sources (e.g., the N LED sources channels; the laser diode channel) and can be configured to detect and report on the spectral properties of the outputs of the individual sub-sources and on the overall output to possibly be recorded, controlled, and displayed. Information generated by the color sensor can be provided to the micro-processor, which in response, can transmit control signals to the optical emitters of the plurality of sub-sources and the one or more filter motors. For example, the color sensor can comprise one or more power output sensors configured to detect the output of one or more output channels (e.g., one or more of the N LED sources channels and the laser diode channel) and can be operatively coupled to the micro-processor or other electronics (e.g., as shown in FIG. 2A) to provide feedback signals indicative of one or more characteristics of the detected light (e.g., output intensity at the excitation peak of the laser diodes). The micro-processor can be configured to be responsive to the feedback signals by controlling the one or more output channels. In some designs, the illumination device or system can be configured to provide one or more modes the yield illumination having desired spectral characteristics. In some cases, the illumination device or system includes a user interface through which the user can select between a plurality of such modes.

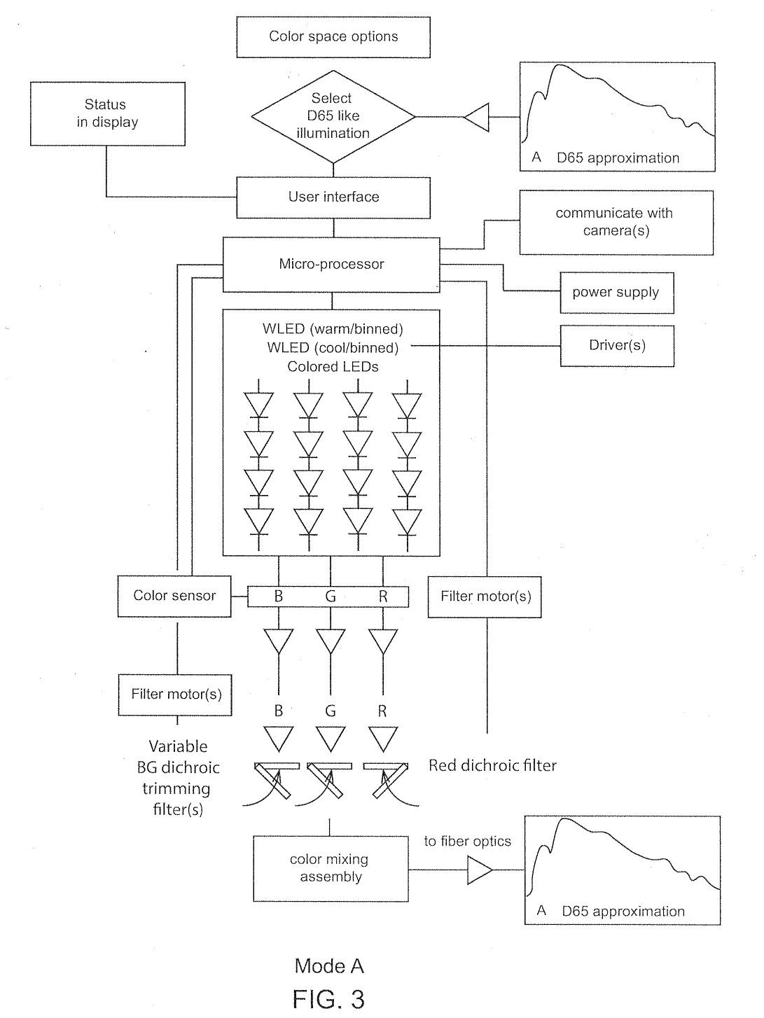

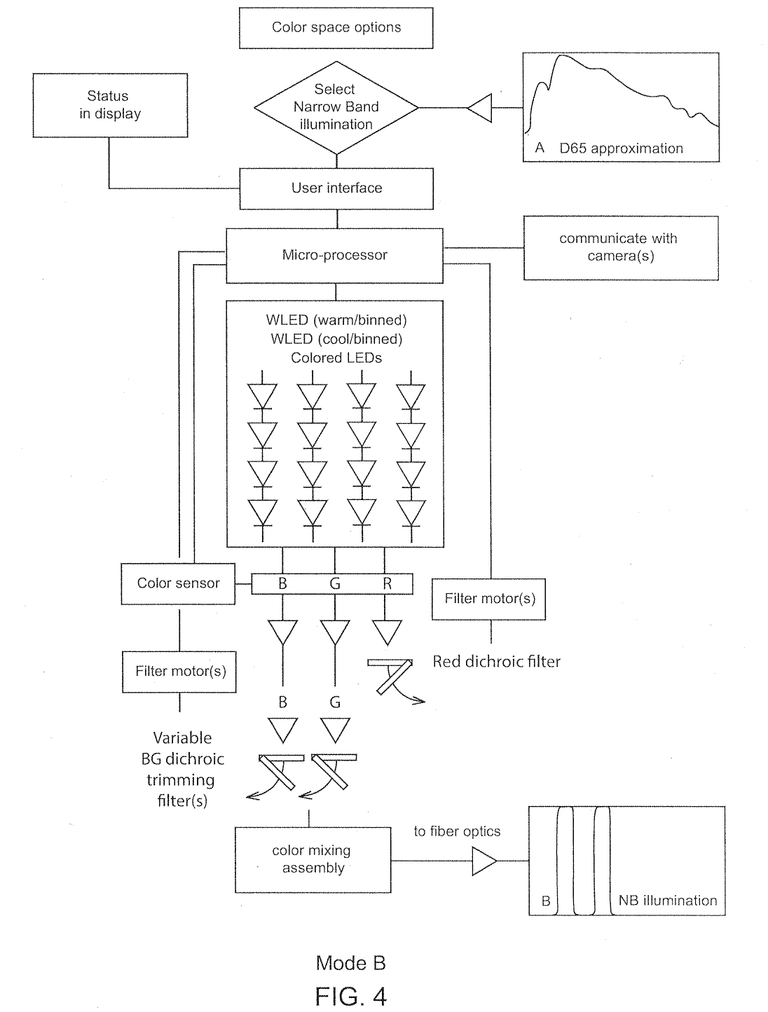

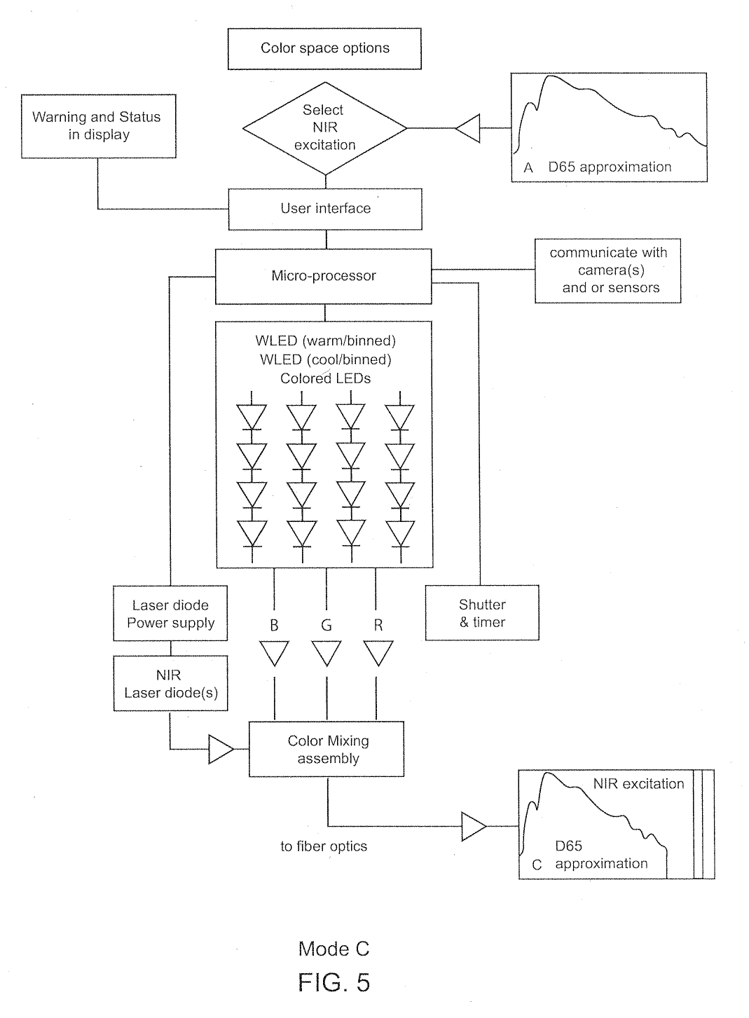

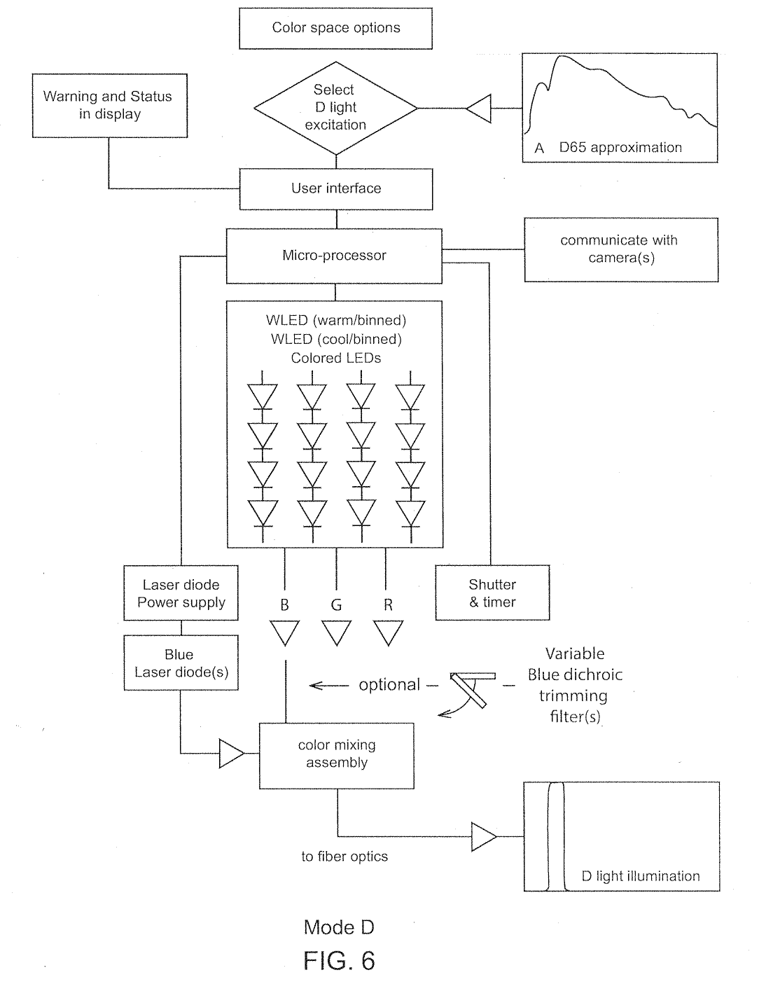

FIGS. 3-6 schematically illustrate example illumination modes which can be offered to the user in a user interface which communicates with one or more displays and one or more cameras.
FIG. 3 illustrates an example Mode A that provides an illumination output that approximates CIE illuminant D65, and that approximates the illumination output of a xenon lamp. In addition, by adjusting power and filter configurations of the example light source, different color balances can be achieved. As illustrated, the illumination system includes blue green dichroic filters and red dichroic filters that having spectral characteristics that can be gradually tuned by varying the orientation of the filter with respect the incident light beams. In addition to the blue green dichroic filters and red dichroic filters, a band stop filter can be used to extinguish the red channel.
FIG. 4 illustrates an example Mode B that provides an illumination output that approximates an illumination output for narrow-band imaging (NBI). For example, the wavelength range for NBI can comprise one or more of the following: blue waveband (e.g., 440 nm-460 nm; range centered at 415 nm and having width of about 10 nm, 20 nm, or 30 nm; range having width greater than 10 nm, greater than 20 nm, greater than 30 nm, less than 10 nm, less than 20 nm, less than 30 nm); green waveband (e.g., 540 nm-560 nm; range centered at 540 nm and having width of about 10 nm, 20 nm, or 30 nm; range having width greater than 10 nm, greater than 20 nm, greater than 30 nm, less than 10 nm, less than 20 nm, less than 30 nm). In addition, the illumination output can advantageously be transitioned from visual imaging to NBI under the user's control. As illustrated, the illumination system includes blue green dichroic filters and red dichroic filters that having spectral characteristics that can be gradually tuned by varying the orientation of the filter with respect the incident light beams. In addition to the blue green dichroic filters and red dichroic filters, a band stop filter can be used to extinguish the red channel.
FIG. 5 illustrates an example Mode C which provides an illumination output which approximates CIE illuminant D65 plus NIR excitation.
FIG. 6 illustrates an example Mode D that provides an illumination output that approximates a blue light only. For example, the Mode D light can comprise a narrow band of blue light (e.g., short wavelength blue light, preferentially absorbed at the tissue surface; approximately one-half of the blue waveband component of NBI; 440 nm-450 nm; 450 nm-460 nm). As another example, the Mode D light can comprise blue illumination as used in photodynamic diagnosis (PDD) of lesions (e.g., flat lesions; epithelial lesions; carcinomas), e.g., in cystoscopy, urology, gynecology, gastroenterology.

In one example, Mode A as shown in FIG. 3 can be selected by the user, and the following example sequence of actions can be performed by the example light source shown in FIG. 2A:
Initiate current to a color sensor with driver currents to the LEDs off, setting the black point (e.g., zero RGB values).
Initiate current to an array of warm and cool white LEDs.
Use color sensor to measure luminance value Y in beam path proximal to fiber optic cable at maximum drive current. Dimming can be a percentage of this value, for example, a lesser value may be used for maximum to account for lifetime changes, and Mode B can utilize a driver current that is raised from that of Mode A.
Read value and store.
Assign warm LED to red-difference chroma components of the color space (CR) in color sensor, read and store value.
Assign warm LED to blue-difference chroma components of the color space (CB) in color sensor, read and store value.
Compute color values of color space for nominal position (setting white balance) according to RGB values in matrix to find white point. Read and store values.
Measure RGB values in CIE xyY space. These values can be derived by integrating the CIE color matching functions with the measurements of spectral power distribution.
Initiate current to an array of colored LEDs (Cyan, Green, Amber, Deep Red, etc.) (each discrete LED color is given a PWM channel to control driver current).
Remeasure RGB values in CIE xyY space.
Compare resulting CIE xy values with D65 in LUT (look up table).
Raise or lower colored LED channel driver current to approximate D65 as stored in LUT.
Results in nominal balanced white light configuration, Mode A.
Report color sensor values to user interface for display or storage.

In another example, Mode B as shown in FIG. 4 can be selected by the user for narrow-band imaging, and the following example sequence of actions can be performed by the example light source shown in FIG. 2A:
Use Mode A for starting point.
Apply current to red filter motor, which tilts the red dichroic filter in the warm and cool white LED beam paths, thereby reducing the red light output. Optionally, an iris diaphragm, band stop filter, and/or neutral density filter (e.g., a rotationally variable neutral density filter, variable in steps or continuously variable) can be used to control the red light channel originating from white light LEDs, which would utilize a different physical layout.
Simultaneously apply additional current to the warm and cool white LED PWM driver channels, thereby raising light output in the remaining blue and green beam paths.
Simultaneously apply additional current to the cyan and green LED PWM driver channels, thereby raising light output in the blue and green beam paths. In certain embodiments, violet and blue could be added as well.
Initiate current to tilting a blue green trimming filter motor which tilts the filter in the beam path and sharpens cut-off and cut-on blue and green wavebands.
Optionally, raise or lower current to LEDs drivers to raise or lower the blue and green output independently. In certain embodiments, violet and blue could be added as well.
Report color sensor values to user interface for display or storage.

In another example, Mode C (e.g., which can be available as an additional modality to either Mode A or B), as shown in FIG. 5, can be selected by the user for NIR excitation added to D65-like illumination of Mode A, and the following example sequence of actions can be performed by the example light source in FIG. 2A:
Select in response to the user interface either the NIR laser diode or the NIR laser diode options.
Display warnings for eye safety in display.
Apply current to NIR laser diode or select from NIR laser diode options to initiate.
Communicate to the one or more cameras that NIR excitation energy is to be used and what type (e.g., 700 nm type or 808 nm ICG type) source is to be initiated. In certain cases, the one or more cameras will have modes or filters for source type (e.g. excitation blocking filters).
Report output value, in radiant flux or current, and source type to user interface and display.

In another example, Mode D, as shown in FIG. 6, can be selected by the user for short blue light for photodynamic therapy (e.g., using photosensitizing agent excitation), where the excitation waveband is in the deep blue and emission in the 625-700 nm range, and the following example sequence of actions can be performed by the example light source in FIG. 2A:
Select in response to the user interface either the blue laser diode or the blue laser diode options (e.g., excitation sources in the 405 nm range).
Display warnings for eye safety in display.
Apply current to the blue laser diode or select from the blue laser diode options to initiate.
Communicate to the one or more cameras that the blue excitation energy is to be used and what type (e.g., 405 nm or Sorel type with Q range options) source is to be initiated. In certain cases, the one or more cameras will have modes or filters for source type (e.g., shutter and timing communication between illuminator and camera).
Report output value, in radiant flux or current, and source type to user interface and display.

The controller subsystem can be configured to allow the user to choose one or more modes or combinations of modes, including those shown in FIGS. 3-6 as well as other modes. For example, NIR excitation could be added to the NBI of Mode B or to Mode D Likewise, the controller subsystem can comprise one or more transition filters which may allow the user to selectively switch among one or more modes, including those shown in FIGS. 3-6 as well as other modes. For example, the micro-processor and one or more filters of the controller subsystem can be configured to allow the user to controllably fade the illumination output from Mode A to Mode D and back.

FIGS. 3-6 also schematically illustrate examples with one or more filters that can be tuned to gradually change the spectral characteristic of the light after interacting with the tunable filter. As discussed above, various filters may comprise one or more stacks of (e.g., dielectric) layers, which may act in a band pass manner by optical interference, rather than absorption, to shift and/or attenuate the spectral power distribution. Certain examples described herein advantageously allow the light source to illuminate various biologically important features of the patient by varying the positioning of the filters through angle space so that the incident angle of light in the illuminator changes with respect to the filter stack. The dielectric stack can be a periodic layering of materials with high index of refraction, such as titanium dioxide (n=2.4) or zinc sulfide (n=2.32), and low index materials, such as magnesium fluoride (n=1.38). The physical thickness of these material layers can be configured to produce a predetermined optical path difference due to the differing indices. Alternately, ordered layers with high, low and medium index materials can be utilized to both pass desired wavebands and attenuate other wavebands and/or to shift the central wavelength (CWL) region to shorter wavebands. In certain embodiments, the filters are tilted through prescribed angles in a collimated beam.

### Monitoring Light Emitters in Real Time During Use

Multiple video cameras can have associated illumination sources whose combined illumination output (e.g., from multiple illuminators) may impact the surgeon's view selection(s) and/or viewing. Sampling of the components such as light sources (e.g., selectively sampling individual or groups of emitters such as lasers or LEDs) within the lighting system may be desirable. Accordingly, in various example configurations, one or more retractor cameras or one or more endoscopes may utilize a lighting system made up of discrete sources such as lights associated the one or more retractor cameras, the one or more endoscopes or other lights sources such as a light source on a display, on a surgical microscope camera, or a stand-alone light source.

Light Emitters (e.g., LEDs) can have varied and inconsistent spectral output depending on process, binning, and other factors. These characteristics can be calibrated with specific spectral output requirements at build time. However, individual light emitters that are fully characterized can still have varying spectral output depending, for example, on power level, temperature, and age.

For applications in which specific spectral output is required (for instance, a D65 broadband white light source), it can be desirable for the additional output variations associated with power level, temperature and age to be calibrated in real time, during the life of a particular product. Further, color profiles associated with various forms of image sensors, 3-chip cameras with dichroic bandpass filters, or single sensors with Bayer filter arrangements, and different camera manufacturer's profile adjustments for providing images of a surgical site may involve further tuning of the individual camera/light source combinations in a surgical visualization system to match physician preferences, choices or modalities, and system component aging.

In various implementations, LEDs can be selected, actuated, and controlled such that the desired spectral output can be targeted appropriately with an adequate combination of varying wavelength LEDs. For instance, for D65 output, a combination of cool white, warm white, cyan, and green LEDs can achieve a D65 output with proper adjustment. However, addressing variations in the LEDs, over power, temperature, and age can further involve frequent fine tuning.

One approach for monitoring and providing adjustment can be to utilize a spectrometer to monitor the light output and then make dynamic adjustments to the light emitter outputs. In various implementations, a spectrometer can be used to monitor one or more light emitters continuously or intermittently. For example, a spectrometer can measure a spectral output (e.g., variation in light intensity, power, output, etc. for different wavelengths) of one or more light emitters and/or differences in the output. In some designs, a spectrometer can be used to measure a group of light sources. For example, spectral output from a group of sources directed to a filter (e.g., blue sources to a blue filter, green sources to a green filter, and/or red sources to a red filter) can be measured. The spectral output can be adjusted using one or more adjustable plates with a transmissive and/or reflective filter. In some implementations, pulsing can be used to measure and/or control one or more light emitters. In some instances, pulse-width modulation can be used.

Another approach can utilize a photodetector configured to measure spectral contribution of one or more light emitters. For example, in some implementations, a distributed surgical lighting system can include a plurality of light emitters, a processor, and a photodetector. The light emitters can be configured to produce a spectral output. The processor can be configured to control an amount of electric current from a power source to drive different light emitters to emit light. Different emitters, for example, can be selectively provided with different amounts of power. The photodetector (e.g., a photodiode) can be configured to measure the spectral contribution of one or more light emitters when other light emitters are not emitting light. A photodetector can have an intrinsic wavelength spectrum and/or be accompanied by a spectral filter (e.g., an absorptive or interference filter).

In order to determine the spectral contribution of the different light emitters, the spectral shape for the light emitters can be characterized separately or in a group and the total optical power output of the specific light emitter(s) at a given time can be observed. This information can be used to calculate appropriate spectral contributions based upon observed output power for the light emitters. To determine the output of one or more specific light emitters, a photodetector can be used. For example, the output of a light emitter can be determined with a photodetector when only that light emitter is turned on and the other light emitters in the system are turned off. As another example, the output of a portion of light emitters can be determined with a photodetector when only that portion of light emitters is turned on and the other light emitters in the system are turned off. In some instances, the lighting system can include a timing circuit. The timing circuit can be configured to reduce or interrupt the power provided to all but one or a portion of the light emitters. The photodetector can measure the spectral contribution of the one or portion of the light emitters emitting light. The timing circuit can repeat this for each or other portions of the light emitters.

If the system, on occasion disables all light emitting channels except one (or a portion) of the light emitters and subsequently scans through all light emitting channels, it is possible to determine the optical contribution of one (or a portion) of the light emitters in the system in real time. In some instances, the system can be configured to disable light emitters for a duration within the range of about 5 ms to about 150 ms. For example, the duration can be about 5 ms, 10 ms, 15 ms, 20 ms, 25 ms, 50 ms, 75 ms, 100 ms, 125 ms, 150 ms, or any range formed by any of these values such as 5 ms to 100 ms, 5 ms to 125 ms, 10 ms to 100 ms, etc. or may be outside any of these ranges. In a live viewing application, it can be undesirable to have visible flicker as light emitting channels are enabled and disabled. As such, it can be important to have light emitters switch times exceeding the visible flicker frequency of the human eye as well as the noticeable responsiveness of a single camera capture frame. To accomplish this, in some implementations, the timing circuit can be configured to reduce or interrupt power for all but one or a portion of light emitters (e.g., all but one or a portion of light emitters is turned off) for a fraction of a video frame (e.g., of the cameras imaging the surgical site) such that resulting intensity changes are not visible to the human eye. In some examples, the fraction of a video frame can be within the range of about 0.5% to about 10%. For example, the fraction can be about 0.5%, 1%, 1.5%, 2%, 2.5%, 3%, 4%, 5%, 7%, 10%, or any range formed by any of these values such 0.5% to 7%, 0.5% to 5%, 0.5% to 3%, etc. or may be outside any of these ranges. Video rates may for example be 50 Hz or 60 Hz for analog video or 120, 240, or 300 frames per second for digital video, however, other rates may be employed. High speed light emitting control loops in excess of 50 KHz can be used in some instances. In some examples, the timing circuit can be configured to reduce or interrupt power for all but one or a portion of light emitters for a duration within the range of 5 ms to 150 ms. For example, if video frames at 50 Hz or 60 Hz for analog video or 120, 240, or 300 frames per second for digital video are used, and LEDs are disabled for a duration of 100 ms, sufficient LED power information can be captured for each channel or a group of channels via a photodetector. This information can be used to in a feedback loop to adjust the power output of one or more light emitters (e.g., one or more LEDs) to achieve a desired spectral output.

In some implementations, the processor can be configured to control light emitted from one or more light emitters based on the measured spectral contributions of the different light emitters to adjust the spectral output of the light emitters. For example, the information captured by the photodetector can inform the LED driver controller of desired LED channel power output to maintain an ideal spectral output (such as D65). Proportional-integral-derivative (PID) control loops can also be used. In some instances, the processor can be configured to control one or more light emitters. In some instances, the processor can be configured to control one or more tuning filter (e.g., tiltable interference filters) associated with one or more light emitters.

In various implementations, the distributed lighting system may be configured to determine spectral contribution of one or more light emitters before and/or after light is modified (e.g., after light is combined, before entering a tiltable filter, after exiting a tiltable filter, etc.). In some instances, the distributed lighting system may include a control unit such that the spectral contribution of one or more light emitters can be determined and/or the spectral output of the light emitters can adjusted. Such activities may occur upon user control. In some implementations such monitoring and/or adjusting of the emitters may be occur after or upon activation by a user of a function such as switching between cameras (e.g., by depressing a button, toggling a switch, using a voice command, etc.). In some instances, the distributed lighting system may be configured to determine spectral contribution of one or more light emitters and/or adjust the spectral output of the light emitters automatically (e.g., after a period of time, when switching views, etc.).

In various implementations, a method to determine a spectral contribution of one or more light emitters in real time is provided. The method can include (a) emitting light from one or more light emitters when other light emitters in the plurality are not emitting light, (b) measuring a spectral output of the one or more light emitters emitting light with a photodetector, and (c) repeating (a) and (b) for another one or more light emitters. In some instances, a method of adjusting a spectral output of a plurality of light emitters is also provided. The method can include determining the spectral contribution of one or more light emitters in the plurality of light emitters as described herein, and controlling light emitted from at least one light emitter based on the determined spectral contribution of one or more light emitters.

### Multiple Light Sources

Various implementations can include a surgical visualization system having one or more cameras. In some implementations, a camera can include a first illumination source (e.g., its own illumination source). The system can also include a second illumination source. The camera can be configured to receive light from each of the first and second illumination sources. For example, the system may include at least one camera, which may include one or more cameras configured to provide a surgical microscope view, one or more endoscopes, one or more cameras on a retractor, one or more cameras on surgical tools, one or more proximal cameras, or one or more exoscopes. The camera can receive light from both of the first and second illumination sources. In some instances, the second illumination source can be an independent illumination source (e.g., not a part of the camera). In some instances, the system can include a second camera comprising the second illumination source. The system can include multiple illumination sources configured to emit light to each of multiple cameras. The illumination sources can be different from other illumination sources. For example, a first illumination source may include a white light source, and a second illumination source may include a colored light source. One or more cameras can be configured to receive light from the white light source and from the colored light source. Other example light sources and combinations are possible. Advantageously, a camera may utilize multiple light sources. In some designs, switching between cameras can be enhanced by light monitoring circuits to avoid or reduce over-saturating the images of cameras positioned at different distances from the surgical site and sources so that the switched view can appear seamless with respect illumination levels.

### Examples of Aggregating Light Sources

Various implementations can use discrete light sources, such as solid state lighting (SSL) sources, and aggregate them to resemble a broadband continuous source, like a Xenon arc, D65, D55, D50, or other illuminates, for example. Some designs can also be configured to switch illumination modes. The Xenon arc typifies many surgical illumination schemes. An arc has a spectrum similar to that of sunlight. In addition, positive features of an arc is its intensity and small size. The arc's drawback can include cost and short life span, including in some cases catastrophic failures. White light LEDs alone, in particular, may suffer in their ability to mimic sunlight or Xenon arc. The radiometric calculation of the product of the area of the light source and the solid angle resulting from the source (e.g., A*Omega) generally tends to favor Xenon arc. Various implementations described herein can improve the output and flexibility of SSL. Various designs can utilize dichroic filters to superimpose light sources or collections of sources over each other by passing a wave group through the filter and when the filter is places at a 45 degree angle, passes another different wave band group. The superposition of waveband outputs can be gathered with fiber assemblies. Superimposing sources can improve the efficiency by reducing the Area of the source, the A in the A*Omega product.

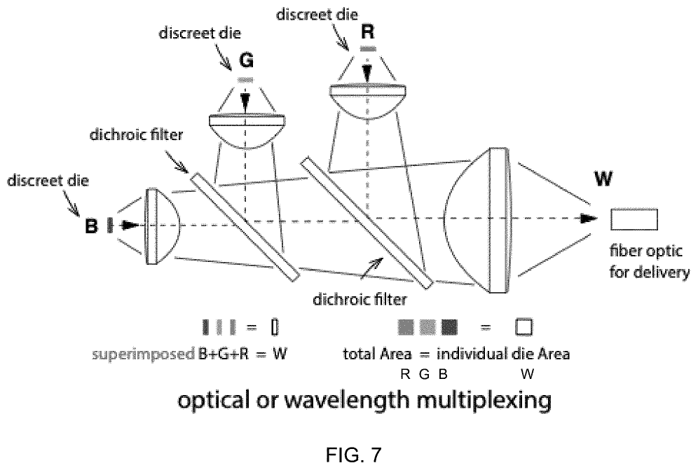

FIG. 7 schematically illustrates an example assembly/method to aggregate discrete light sources (e.g., of blue B, green G, and red R light) into a fiber optic bundle, which can be used to deliver light (e.g., the combined white light) to a surgical site whether through a retractor camera, endoscope, exoscope, microscope or camera on a tool. In FIG. 7, the shortest wavelength, blue light in this case, is passed on axis to a dichroic filter (beam combiner) which transmits blue light, but reflects green light at a 45° angle. The combined blue green combination passes to another dichroic filter (beam combiner) which reflects red at a 45° angle and the combined red, green, and blue light combine to form a broadband source, e.g., white light.

Some implementations can be used to combine light from high powered large LEDs into larger medical fiber optic cables. Some examples may be used with a surgical microscope camera. In some instances, the elements can be rather large, e.g., individual elements can be 12, 15, 20, 22, or 25 mm to 50 mm. Some such implementations can be acceptable in a control box that sits on a cart near the surgical table, but may not allow the sources to be near the patient in some cases. Contributing sources may be collimated prior to combining. The collimation optics or collimator (e.g. collimation lens) can introduce chromatic aberrations in addition to spectral variations in sources. Fibers and apertures can help in various instances. In addition, the fiber optic cable can deliver light to the microscope or surgical microscope camera and stand.

In some examples, the spectrum may not be quite like a Xenon, D65, D55, D50, etc., when R G and B only are used. In addition, while additional wavelength LEDs, like Cyan, Amber and Red, can be added, tailoring the light output may be a challenge. LED sources are generally measured by their manufacturers by peak wavelengths and half max height, both of which can vary by manufacturer, by age of the die, by lot, etc. Characterizing the output of each source at the factory and accounting for aging and variability may not be straightforward. In various implementations, an integrated spectrometer or other method of measuring the output spectrum (e.g., intensity or output vs. wavelength variations or distribution) can be used. In addition, some designs can utilize tilting plates with dichroic coatings as described herein to adjust the spectral distribution (possibly in response to the spectral measurements). By using controllable tilting plates with dichroic coatings applied to their surface(s), unwanted peaks from SSL sources can be reduces or shifted to more closely conform to the light output of a desired illuminant, such as D65, D55, D50, or other desired output. For example, D50 output is less blue and warmer than a D65 output. D50 has more extended red output which can be useful in differentiating between arterial and venous blood flow.

Tilted plates can also re-direct one or more SSL sources to a fiber optic or light guide assembly. As described herein, various implementations can include fiber optics with each input and output end being a different shape. Microscope systems with an integrated SSL system can be produced utilizing single large LEDs, which in the past may use a large collimating optic, in some implementations by splitting into small groups so that the collimating optics are smaller and fit with an unobscured view. Small LED die are typically square which can be challenging to optically couple to fiber optics. Efficiency can be gained utilizing better packing through adjacencies (e.g., the reduction of source A is accomplished by using fiber optics to combine one or more rectangular sources into a circular output for efficient fiber coupling). In some designs, the output of a large LED can be coupled to a group of fibers or light guides which can be split into a number of output fibers, whose faces can be oriented to reduce vignetting in a system looking into a cavity.

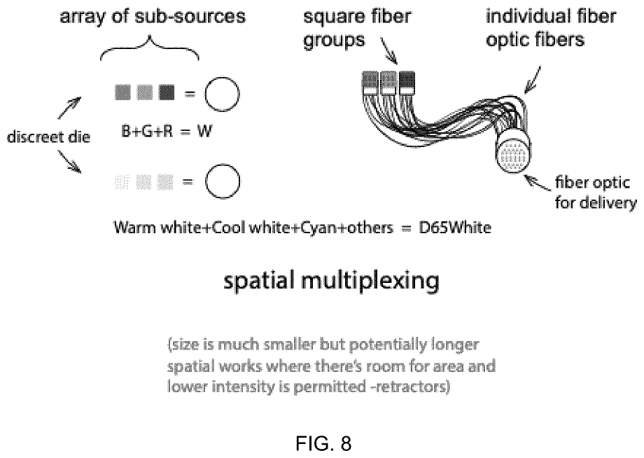

FIG. 8 schematically illustrates another example assembly/method of aggregating discrete sources. For small LED die sizes, square fiber optic bundles can be placed in close proximity to the die and optically coupled without lenses. This can be very efficient when the Numerical Aperture of the fiber is high, 0.66NA or 0.83NA or 0.87NA for example. The fibers can be randomized in the loose central section (e.g., between the fiber ends), gathered at the output end, epoxied, cut, and polished. The shape can be round and an efficient way of transforming square sources into round output. Alternately, a light pipe assembly can involve fibers being replaced by solid plastic or glass combining the square output of the LED dies and producing a circular output. Other cross-sectional shapes are also possible.

As described herein, discrete sources can be aggregated to approximate a broadband spectrum. Wavelength multiplexing (e.g., FIG. 7) can increase and/or maximize the amount of radiant power transferred from a source to a second location (e.g., to a fiber delivery system), at the expense of expense and size of components in some instances. The flux or radiant power output advantage can be the result of superimposing each die over one another. In some instances, the spatial multiplexing (e.g., FIG. 8) can be small in size and low in cost. The aggregating assemblies/methods (e.g., wavelength and/or spatial) can feed a fiber optic cable, utilized for its flexibility, to the instrument, typically some distance away from the light source. It can be done directly from the optical output or fiber optic.

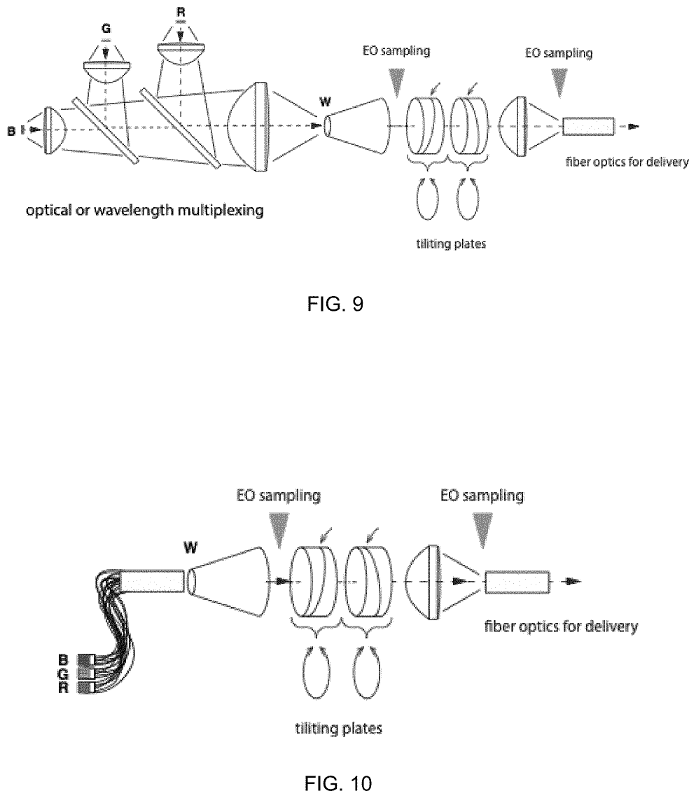

As described herein and schematically illustrated in FIGS. 9 and 10, for further refinement of the spectrum, the aggregated sources can provide flux to a tilting plate(s) with dichroic filters. The EO (electro-optical) sampling can be a method where all but one of the LEDs are turned off and a single LED is sampled by a photodetector (or spectrometer). Analog video can be 50 Hz or 60 Hz. Digital video standards can be 120, 240, or 300 frames per second. The timing of the LED sampling can be a small fraction of a video frame rate, for example 1%. The short duration of flux reduction, for example, 10 ms, 50 ms or 100 ms, may not be noticed by the viewer (e.g., due to persistence of vision).

Using control and/or processing electronics, the (e.g., EO) sampled output of the single LED can be compared with its original output in a look up table (e.g., compiled at manufacture) and its sampled contribution to the total spectrum can be adjusted relative to the measured power of the remaining LEDs sampled in similar fashion, resulting in a current state of the spectrum. When a tilting plate mechanism or other tunable filter is added to the system, a second (e.g., EO) sampling point may be added to measure in similar fashion the tailored output of the adjusted system (as illustrated in FIGS. 9 and 10).

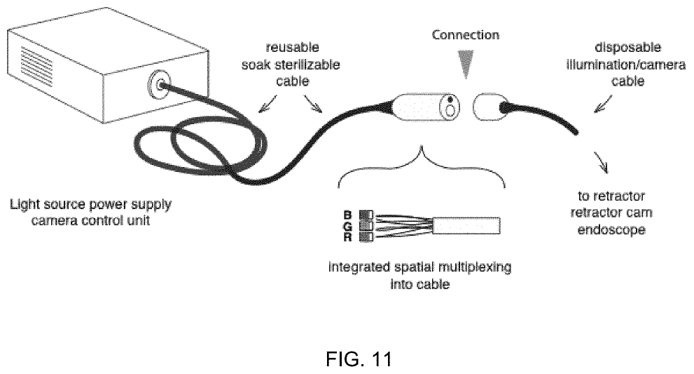

As schematically illustrated in FIG. 11, in some spatial multiplexing designs, the small size may permit the LEDs and fiber aggregating system to be integrated into a fiber optic delivery cable made of two parts. A first reusable, soak sterilizable, section of as much as 2 meters in length. The first part of such a cable may connect to an integrated camera control unit/illuminator. The spatial multiplexed implementation can be integrated into the distal end of the reusable cable, the connector can become the heat sink. It is shown without an optional tilting plate mechanism. The disposable second portion of the cable can go to the instrument (e.g., camera on retractor, endoscope, etc.).

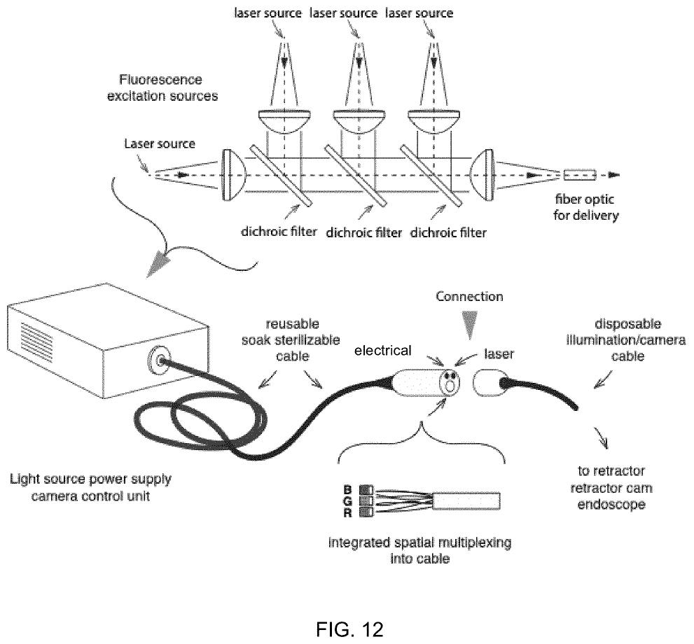

As shown in FIG. 12, further implementations can include wavelength multiplexing for excitation laser sources into the camera control/illuminator box. An additional fiber connection in the two part cable can bring laser sources to a disposable distal assembly. Either multiplexing using beamcombiners such as dichroic mirrors, or multiplexing using optical fiber or both may be used depending on the implementation. For example, one may provide broadband illumination and the other may provide narrowband, for example, excitation for fluorescence.

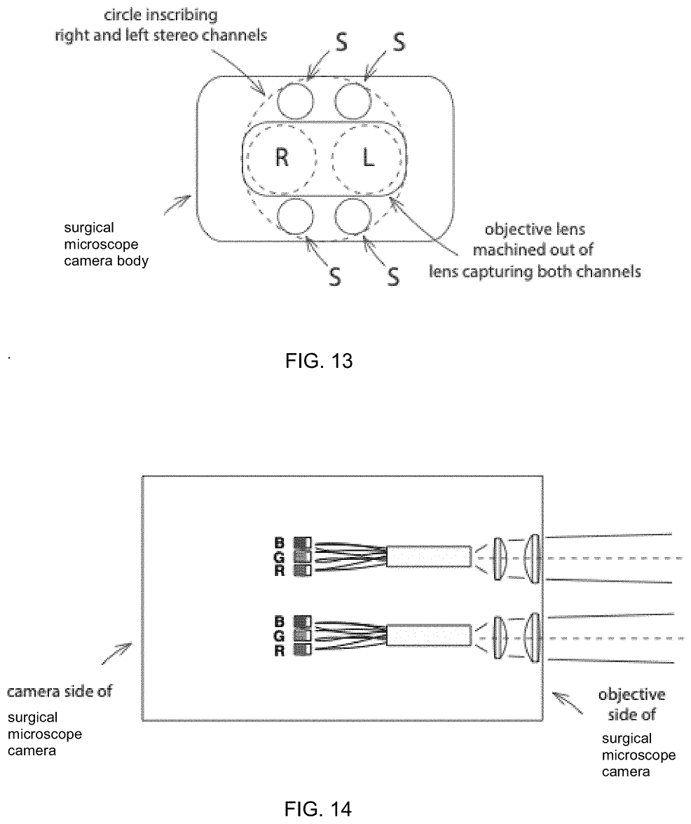

As shown in FIG. 13, in some implementations, a digital stereo camera with an objective lens can incorporate, 1, 2 or 4 spatial multiplexing engines. In some cases, the orientation of the multiplexers can be on the top and bottom of the R and L eye channels. Further, to reduce and/or avoid multiple reflections from illumination returning to the cameras through the zoom lenses, the objective lens, shown as a large dashed line circle can be cut down to permit separate illumination paths so there is reduced and/or low and/or no cross talk with a shared objective. The sources, marked S, can be within the inscribed circle to reduce and/or minimize vignetting from the surgeon's hand, instrument, or slight misalignment to the surgical site, which can often be in a recess, port, opening, or opening with a retractor.

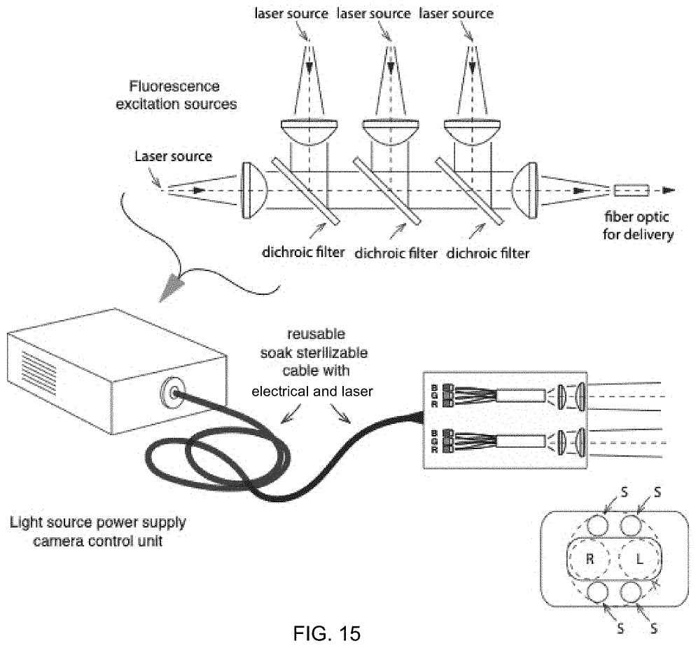

FIG. 14 is a top down view of example multiplexers on a surgical microscope camera or on a stereo camera for viewing surgical sites. FIG. 15 schematically illustrate an example surgical microscope with an illuminator. A multiplex including a plurality of beam combiners (e.g., dichroic filters) is used in combination with fiber multiplexers. In some example implementations, the former multiplexer includes laser sources and may possibly be used for narrow band illumination such as for fluorescence excitation while the fiber multiplexer may comprises LED (e.g., R, G, B LEDs) and may be used for broadband (e.g., visible) illumination for the surgeon to see the surgical site. Either or both may be used. Different combinations of light sources and multiplexers are possible. Multiple outputs of the multiplexers and/or light sources may be included and may be disposed on opposite sides of the left and right stereo channels and/or disposed about the channels in different arrangements.

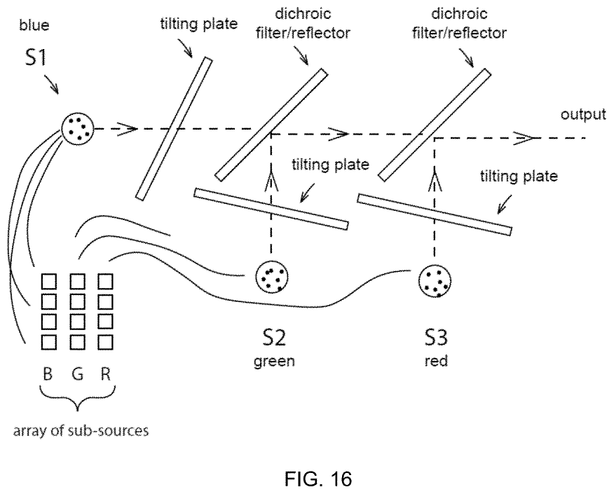

FIG. 16 schematically illustrates another example multiplexing assembly/method. In FIG. 16, a combination of light sources S1, S2, and S3 are shown as Blue, Green, and Red sources, for example, with the shortest wavelength (blue) farthest from the output. The rectangular die making up the sources S1, S2, and S3 are collected by square ended fibers or light guides coupled from the rectangular die and at an arbitrary distance redistributed as circular faced outputs at the positions of S1, S2, and S3. This is an example of addition by adjacency effectively making N number of rectangular dies appear as a single source, and circular faced in this implementation. The distal fiber end could be further coupled to tapers (e.g., tapered lightguides) or lenses to collimate the output prior to the tilting plates (e.g., tunable filters). Tapers can reduce the Numerical Aperture of a source when oriented with the smaller end toward the source and the larger end toward the output end. Tapers can act as a mixer to help homogenize the output of an SSL alone or in combination with lenses. In some instances, lenses alone may collimate, but may not mix light output from one or more sources possibly depending on the configuration. The tilting plates, which when tilted, can shift or trim unwanted peaks, for example, from the combined output of S1, or the other sources S2, S3. The tiltable plates can be an addition to any electrical communication and control which reduces or increases the output of one or more of the constituent die sources. Short pass, or long pass dichroic filters (beam combiners, or beamsplitters), depending on the orientation of the source order to output, are shown at 45 degrees relative to the optical axis, and made up of coating layers applied to plane parallel plates. These dichroic filters or beam combiners can be tilted at different angles other than 45 degrees in other implementations. Other types of beam combiners may also be used. In this example, the dichroic filters (e.g., long pass filters) pass the shorter wavelengths of S1 (e.g., blue sources) and reflect the output of S2 (e.g., green sources) and S3 (e.g., red sources) respectively. The output of S1, S2, and S3 are superimposed over one another, reducing the Area of the total source and raising efficiency. This is an example of superposition of sources that can improve the output by reducing the Area in the A*Omega product of the summed sources. Other examples are possible.

In some designs, sampling by electrical and/or optical sampling can measure the output of individual or a group of sources, and the output can be adjusted. One or more photodetectors and/or spectrometers can be included to measure the light output (e.g., intensity, power, etc.). In some configurations, the measurements can be performed on the light beam from the light sources prior to being incident on the beam combiners. The amount of each LED's power to vary can depend at least in part on whether its contribution to the total desired illuminate and whether it is above or below the spectral power distribution of the desired illuminate, e.g., D50, D55, D65, or other illuminate. For example, the green channel may be made up of numerous die of varying shades of green (e.g., spectral distributions within the green range). Green LEDs typically have less output power than red LEDs, for example, so in some implementations, numerous green die can be grouped together. Some of the green die can be closer to the blue band and some can be closer to the red band. As shown in FIG. 16, such light can be reflected from one dichroic reflector. The distribution of each color element within this range can be considered one ‘color’ band within the total spectral power distribution. The total spectral output of the system can be measured directly or inferred.

In some instances, an integrated spectrometer can be configured to directly measure, e.g., via a sampling fiber, lens, mirror, or combination of those elements, the total and mixed output of the system near its output end. By sequentially measuring the constituent sources, adjustment could be made to more accurately match a desired output. This can be a direct measurement and its accuracy may depend in part on the sophistication of the integrated spectrometer and/or sampling method.

In some instances, an external spectrometer, such as a bench top unit rather than small integrated unit, can be configured to measure and adjust (and measure and readjust) the constituent sources to produce a desired outcome and the resulting power levels for each source element stored in, e.g., a LUT (look up table) possibly in control and/or processing electronics for application when the user desires that distribution. This can be a factory calibration, and can be considered an inferred result in practice. To take into account the spectral drift or aging of the sources, the LUT could be modified to account for the age of the sources, differences in lots, and/or number of cycles and make adjustments accordingly. The LUT could be also be used in some implementations with an integrated spectrometer as a reference.

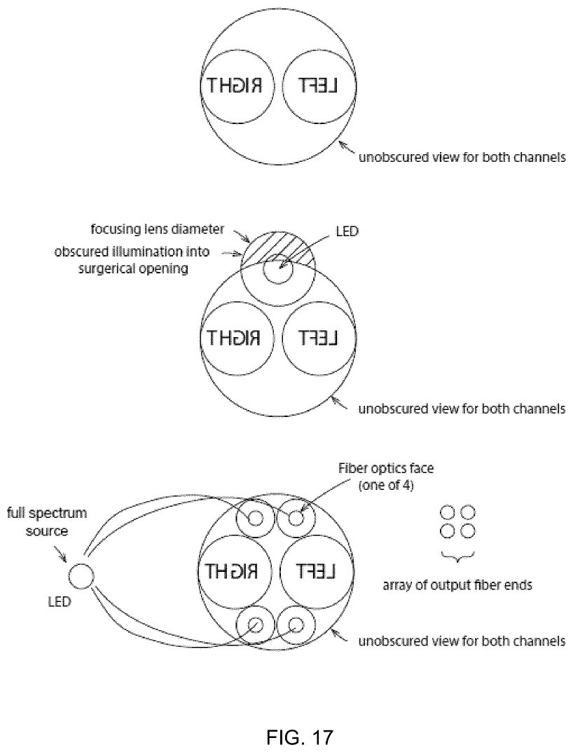

FIG. 17 schematically illustrates an example optical system for imaging a surgical site (e.g. a proximal camera, a camera that provides surgical microscope views, a surgical microscope) that can benefit by utilizing a distributed fiber optic or light guide assembly. FIG. 17 is a view from the position of the surgical site towards the camera (e.g., stereo camera, proximal camera, surgical microscope camera, operating room microscope, etc.). The upper part of FIG. 17 schematically illustrates the left and right channels of the stereo optical system, for example, the left and right imaging pathways of the camera. The circles represent the outer edges of the view by each eye or camera. The minimum circle for an unobscured view in both channels of the camera is also shown.

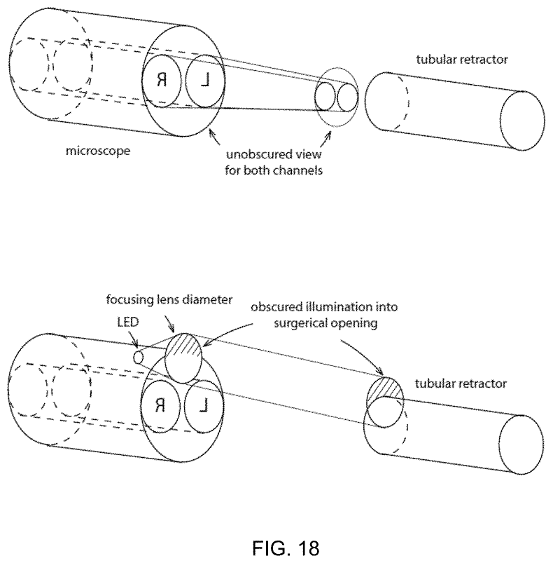

The middle part of FIG. 17 schematically illustrates a single LED and its projection or focusing lens added. A lens sufficient to focus the output of an LED powerful enough to illuminate the surgical site is most often too large to provide unobscured illumination into a deep cavity. Making the lens smaller reduces the power of the source directed onto the surgical site and likely insufficient illumination is produced. Even if the LED and lens are tipped toward the center point between the two stereo channels, there can still be obscuration of the illumination in deep cavities, or when using retractors such as tubular retractors for spine surgery. FIG. 18 is a side perspective view of the example camera shown in FIG. 17. The upper part of FIG. 18 shows the unobscured view in both eye channels (left and right channels of stereo optical system, e.g., camera). The lower part of FIG. 18 shows the obscured illumination into the surgical opening.

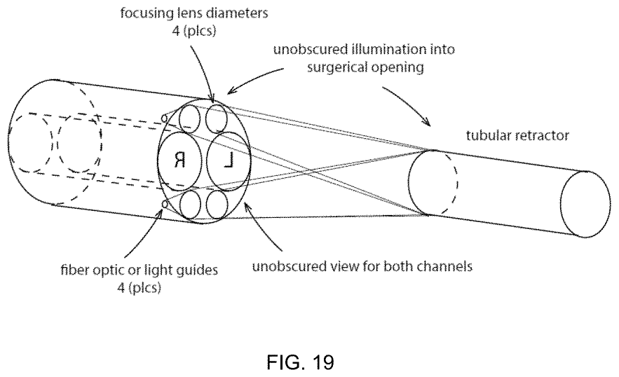

The lower part of FIG. 17 schematically illustrates how the example optical system (e.g., camera) for imaging the surgical site can benefit by utilizing a distributed fiber optic or light guide assembly. The lower part of the FIG. 17 shows a large LED located elsewhere within the optical system (e.g., camera) or adjacent to the optical system/camera and a single fiber or light guide face directly coupled to the LED. The output ends of the fiber or light guide are divided into N parts, 4 in this case (although 3, 4, or more may be used), and the lenses to focus their output can fit within the imaging circle providing the surgeon with an unobscured and fully illuminated view. Dividing the larger source by fibers or light guides can keep the focusing lenses within the imaging circle. For example, the divergence of light from the fiber can be smaller than for the LED and hence a smaller lens can be used. The smaller lens can fit in the camera aperture/housing without having a portion thereof obstructed. FIG. 19 is a side view of the example camera shown in the lower part of FIG. 17. FIG. 19 shows the unobscured view in both eye channels and the unobscured illumination into the surgical opening. The circles representing the left and right channels and the unobscured view for both channels are in the same plane. In some designs, the LED or the output of the fibers can be inside a microscope or a camera that provides a surgical microscope view or in a proximal camera and the focus lens can be in the same plane as the left and right channels. By dividing the output ends of the fiber or light guides into N parts can help as much light as possible down the retractor. Because tools are also used, e.g., in the retractor, there may be an obstruction. Multiple ports can help reduce and/or avoid the light that is blocked as for a single source. Accordingly, in various implementations, a light guide having an input at a proximal end and outputs at a plurality of distal ends provide illumination to the surgical site. A plurality of lenses may be used, each lenses disposed with respect to a respective one of the distal ends of the light guide to receive light from the outputs for transmission to the surgical site. In various implementations, the light guide can include a fiber optic light guide, a fiber optic bundle and/or a light pipe.

In some other implementations, an array of smaller LED sources could be substituted for the single source.

Any systems, devices, components, and/or features described herein can be combined with any other systems, devices, components, and/or features described herein. For example, any systems, devices, components, and/or features described in connection with FIG. 2B can be combined with any other systems, devices, components, and/or features described elsewhere herein, including with respect to any of the other Figures. Various examples of illumination devices and their methods of use are described herein, such as the examples enumerated below:

### EXAMPLE SET I

### Example 1

An illumination device comprising:
a plurality of sub-sources, each sub-source configured to generate a light beam;
at least one filter configured to controllably adjust a spectral power distribution of at least one of the light beams generated by a corresponding at least one sub-source of the plurality of sub-sources and incident on the at least one filter, the spectral power distribution controllably adjusted to provide a gradual transition or a variable change of the spectral power distribution; and
a color mixing assembly configured to receive the light beams from the at least one filter and to generate a composite light beam,
wherein the at least one filter has a spectral distribution that is altered when the at least one filter is tilted with respect to the light beam incident thereon or vice versa.

### Example 2

The illumination device of Example 1, wherein at least one sub-source of the plurality of sub-sources comprises at least one solid-state semiconductor optical emitter producing monochromatic visible light when an electric current is provided.

### Example 3

The illumination device of Example 1 or Example 2, wherein at least one sub-source of the plurality of sub-sources comprises at least one solid-state optical emitter containing phosphor and producing a white light output.

### Example 4

The illumination device of any of Examples 1-3, wherein all the sub-sources comprise at least one colored LED.

### Example 5

The illumination device of any of Examples 1-4, wherein all the sub-sources comprise at least one laser diode.

### Example 6

The illumination device of any of Examples 1-5, wherein the at least one filter comprises an interference filter.

### Example 7

The illumination device of any of Examples 1-6, wherein the at least one filter comprises at least one plane-parallel plate with one or both surfaces coated with a thin film coating stack comprising a plurality of layers with different indices of refraction, wherein tilting of the plate relative to the light beam transmitted through the plate selectively passes or blocks certain wavelength regions of the light beam.

### Example 8

The illumination device of Example 7, wherein the at least one plate is positioned in a collimated beam path of the light beam.

### Example 9

The illumination device of any of Examples 1-8, wherein at least one sub-source of the plurality of sub-sources comprises at least one optical emitter and at least one pulse-width modulation (PWM) circuit configured to control the at least one optical emitter to improve time variant light quality of the at least one sub-source.

### Example 10

The illumination device of any of Examples 1-9, wherein at least one sub-source of the plurality of sub-sources comprises at least one optical emitter and at least one pulse-width modulation (PWM) circuit configured to control the at least one optical emitter to conform to user requirements for perceptually flicker free illumination.

### Example 11

The illumination device of any of Examples 1-10, wherein at least one sub-source of the plurality of sub-sources comprises at least one optical emitter and at least one pulse-width modulation (PWM) circuit configured to control the at least one optical emitter to provide perceptually flicker free illumination at frequencies substantially above 200 Hz.

### Example 12

The illumination device of any of Examples 1-11, wherein the color mixing assembly comprises a collimator.

### Example 13

The illumination device of any of Examples 1-12, further comprising an optical focusing assembly to converge the composite light beam and to transmit the composite light beam to a receiving fiber optic conduit or cable.

### Example 14

The illumination device of any of Examples 1-13, wherein the plurality of sub-sources comprises at least one sub-source comprising a plurality of light emitters configured to produce light flux, the illumination device further comprising a plurality of optical fibers, each optical fiber of the plurality of optical fibers comprising a first end portion configured to receive the light flux from a corresponding light emitter and a second end portion configured to emit the received light flux, the light emitters arranged in a first pattern, the first end portions arranged in the first pattern, and the second end portions are arranged in a second pattern different from the first pattern.

### Example 15

An illumination device comprising:
at least one sub-source comprising a plurality of light emitters configured to produce light flux; and
a plurality of optical fibers, each optical fiber of the plurality of optical fibers comprising a first end portion configured to receive the light flux from a corresponding light emitter and a second end portion configured to emit the received light flux, the light emitters arranged in a first pattern, the first end portions arranged in the first pattern, and the second end portions are arranged in a second pattern different from the first pattern

### Example 16

The illumination device of Example 14 or Example 15, wherein the at least one sub-source comprises an array of sub-sources.

### Example 17

The illumination device of any of Examples 14-16, wherein the array of sub-sources is arranged in a rectilinear, square, or rectangular first sub-pattern, the light emitters of each sub-source arranged in a rectilinear, square, or rectangular second sub-pattern, the first pattern comprising the first sub-pattern and the second sub-pattern, and the second end portions are arranged in a circular pattern.

### Example 18

The illumination device of any of Examples 14-17, wherein the plurality of optical fibers are mechanically coupled together in at least one first end assembly containing the first end portions and at least one second end assembly containing the second end portions.

### Example 19

The illumination device of Example 18, wherein the at least one first end assembly has a non-round format and the at least one second end assembly has a round format or a different non-round format.

### Example 20

The illumination device of Example 18, wherein the at least one first end assembly has a square format and the at least one second end assembly has a round format.

### Example 21

The illumination device of any of Examples 14-20, wherein the plurality of optical fibers are configured to be moved such that the second end portions are positioned at various selected locations relative to the first end portions.

### Example 22

The illumination device of any of Examples 14-21, wherein the first end portions are tapered.

### Example 23

The illumination device of any of Examples 14-16, wherein the plurality of optical fibers are mechanically coupled together in a plurality of end assemblies containing the first end portions and one second end assembly containing the second end portions.

### Example 24

The illumination device of any of Examples 14-16, wherein the plurality of optical fibers are mechanically coupled together in a plurality of end assemblies containing the first end portions and a plurality of second end assemblies containing the second end portions.

### Example 25

The illumination device of any of Examples 14-16, wherein the first end portions are tapered with respect to the second end portions such that the first end portions are smaller than the second end portions.

### Example 26

The illumination device of any of Examples 14-16, wherein the second end portions are tapered with respect to the first end portions such that the second end portions are smaller than the first end portions.

Additional examples are enumerated below:

### EXAMPLE SET II

### Example 1

A distributed surgical lighting system comprising:
a plurality of light emitters configured to produce a spectral output;
a processor configured to control an electrical signal from a power source to drive different light emitters to emit light; and
a photodetector configured to measure a spectral contribution of at least one of the plurality of light emitters when other light emitters in the plurality of light emitters are not emitting light.

### Example 2

The distributed surgical lighting system of Example 1, further comprising a timing circuit configured to reduce power provided to one or more of the plurality of light emitters at a time such that the spectral contribution of the at least one of the plurality of light emitters can be measured by the photodetector.

### Example 3

The distributed surgical lighting system of Example 2, wherein the timing circuit is configured to reduce power for a fraction of a video frame.

### Example 4

The distributed surgical lighting system of Example 3, wherein the timing circuit is configured to reduce power for 0.5% to 10% of a video frame.

### Example 5

The distributed surgical lighting system of any of Examples 2-4, wherein the timing circuit is configured to reduce power for 5 ms to 150 ms.

### Example 6

The distributed surgical lighting system of any of the preceding Examples, wherein the processor is configured to adjust the spectral output of the plurality of light emitters based on the measured spectral contribution of the at least one of the plurality of light emitters.

### Example 7

A method to determine a spectral contribution of one or more of a plurality of light emitters in real time, comprising:
(a) emitting light from one or more light emitters when other light emitters in the plurality are not emitting light;
(b) measuring a spectral output of the one or more light emitters emitting light with a photodetector; and
(c) repeating (a) and (b) for another one or more light emitters.

### Example 8

A method of adjusting a spectral output of a plurality of light emitters, comprising:
determining the spectral contribution of one or more light emitters in the plurality of light emitters according to Example 7; and
controlling light emitted from at least one light emitter based on the determined spectral contribution of one or more light emitters.

### Example 9

A surgical visualization system comprising:
at least one camera configured to image a surgical site, the at least one camera comprising a first illumination source; and
a second illumination source, wherein each of the first and second illumination sources is configured to emit light to a surgical site imaged by the at least one camera.

### Example 10

The surgical visualization system of Example 9, comprising a second camera comprising the second illumination source.

### Example 11

The surgical visualization system of Example 10, further comprising a third illumination source, wherein each of the first, second, and third illumination sources is configured to emit light to the surgical site imaged by the at least one camera.

### Example 12

The surgical visualization system of Example 11, comprising a third camera comprising the third illumination source.

### Example 13

The surgical visualization system of any of Examples 9-12, wherein the at least one camera includes one or more cameras configured to provide surgical microscope views, one or more endoscopes, one or more cameras on retractors, one or more cameras on surgical tools, one or more proximal cameras, or one or more exoscopes.

### Example 14

The surgical visualization system of any of Examples 9-13, wherein the first illumination source is different than the second illumination source.

### Example 15

The surgical visualization system of any of Examples 9-14, wherein the first illumination source is a white light source, and the second illumination source is a colored light source.

### Example 16

The distributed surgical lighting system of any of Examples 1-6, wherein the system is configured to emit light to a surgical site imaged by at least one camera.

### Example 17

The distributed surgical lighting system of Example 16, wherein the at least one camera includes one or more cameras configured to provide surgical microscope views, one or more endoscopes, one or more cameras on retractors, one or more cameras on surgical tools, one or more proximal cameras, or one or more exoscopes.

### Example 18

The method of any of Examples 7-8, wherein the plurality of light emitters is configured to emit light to a surgical site imaged by at least one camera.

### Example 19

The method of Example 18, wherein the at least one camera includes one or more cameras configured to provide surgical microscope views, one or more endoscopes, one or more cameras on retractors, one or more cameras on surgical tools, one or more proximal cameras, or one or more exoscopes.

### Example 20

A controller in communication with the distributed surgical lighting system of any of Examples 1-6 and with at least one camera configured to image a surgical site.

### Example 21

The controller of Example 20, wherein the at least one camera includes one or more cameras configured to provide surgical microscope views, one or more endoscopes, one or more cameras on retractors, one or more cameras on surgical tools, one or more proximal cameras, or one or more exoscopes.

### Example 22

A controller in communication with the surgical visualization system of any of Examples 9-15, wherein the controller is configured to control the first and second illumination sources when the at least one camera is imaging the surgical site.

### Example 23

The controller of Example 22, wherein the at least one camera includes one or more cameras configured to provide surgical microscope views, one or more endoscopes, one or more cameras on retractors, one or more cameras on surgical tools, one or more proximal cameras, or one or more exoscopes.

### EXAMPLE SET III

### Example 1

An illumination device comprising:
a plurality of light sources, said plurality of light sources including first and second light sources configured to generate first and second light beams respectively;
a first beam combiner configured to receive light from said first and second light sources and combine said first and second light beams to generate a composite light beam; and
a plurality of filters, said plurality of filters comprising a first filter configured to controllably adjust a spectral power distribution of the first light beam incident on the first filter and a second filter configured to controllably adjust a spectral power distribution of the second light beam incident on the second filter,
wherein the first and second filters are configured to be tuned to alter the spectral power distribution of the first and second light beams.

### Example 2

The illumination device of Example 1, wherein at least one light source of the plurality of light sources comprises at least one solid-state semiconductor optical emitter producing colored visible light when an electric current is provided.

### Example 3

The illumination device of Example 1 or Example 2, wherein the plurality of light sources comprise at least one colored LED.

### Example 4

The illumination device of Example 1 or Example 2, wherein the plurality the light sources comprise at least one laser diode.

### Example 5

The illumination device of Example 1 or Example 2, wherein at least one source of the plurality of sources comprises at least one solid-state optical emitter containing phosphor and producing a white light output.

### Example 6

The illumination device of any of Examples 1-5, wherein said first beam combiner comprises a reflector that is partially reflective and partially transmissive.

### Example 7

The illumination device of any of Examples 1-6, wherein said first beam combiner comprises a beamsplitter.

### Example 8

The illumination device of any of Examples 1-6, wherein said first beam combiner is partially transmissive, partially reflective surface to transmit said first beam and reflect said second beam.

### Example 9

The illumination device of any of Examples 1-8, wherein said first beam combiner comprises a dichroic reflector configured to transmit light of a first wavelength included in said first light beam and reflect light of a second wavelength included in said second light beam.

### Example 10

The illumination device of any of Examples 1-9, wherein said first light source is a colored light source and said first light beam is a first color and said second light source is a colored light source and said second beam is a second color different from said first color.

### Example 11

The illumination device of any of Examples 1-10, wherein the plurality of light sources further comprises a third light source configured to generate third light beam.

### Example 12

The illumination device of Example 11, further comprising a second beam combiner configured to receive light from said third light sources and combine said third light beam with said composite light beam.

### Example 13

The illumination device of Example 11 or 12, wherein said third light source is a colored light source and said third beam is a third color different from said first and second colors.

### Example 14

The illumination device of Example 12 or 13, wherein said second beam combiner comprises a reflector that is partially reflective and partially transmissive.

### Example 15

The illumination device of any of Examples 12-14, wherein said second beam combiner comprises a beamsplitter.

### Example 16

The illumination device of any of Examples 12-15, wherein said second beam combiner comprises a dichroic reflector configured to transmit light of a first wavelength included in said first light beam and reflect light of a third wavelength included in said third light beam.

### Example 17

The illumination device of any of Examples 12-16, wherein said second beam combiner is configured to transmit light of a second wavelength included in said second light beam.

### Example 18

The illumination device of any of Examples 1-17, wherein the said first and second optical filters are configured to be tuned by tilting the first and second filters with respect to the respective first and second light beam incident thereon or vice versa.

### Example 19

The illumination device of any of Examples 1-18, wherein the first filter and the second filter each comprises an interference filter.

### Example 20

The illumination device of any of Examples 1-19, wherein the plurality of filters comprises at least one plane-parallel plate with one or both surfaces coated with a thin film coating stack comprising a plurality of layers with different indices of refraction, wherein tilting of the plate relative to the light beam transmitted through the plate selectively alters which wavelengths in the light beam are passed or blocked.

### Example 21

The illumination device of any of Examples 1-20, further comprising a first photodetector disposed to receive a portion of light from said first light source to measure light output from said first light source.

### Example 22

The illumination device of any of Examples 1-21, further comprising second beam combiner, a second photodetector, and a third light source, said second beam combiner configured to receive a third light beam from said third light source and combine said third light beam with said composite light beam, said second photodetector disposed to receive a portion of light from said third light source to measure light output from said second light source.

### Example 23

The illumination device of any of Examples 1-23, further comprising a first collimator disposed to receive light from the first light source and direct said light onto said first beam combiner.

### Example 24

The illumination device of any of Examples 1-23, further comprising a second beam combiner, a second collimator, and a third light source, said second beam combiner configured to receive a third light beam from said third light source and combine said third light beam with said composite light beam, said second collimator disposed to receive light from the second light source and direct said light onto said second beam combiner.

### Example 25

The illumination device of Example 23 or 24, wherein said collimator comprises a lens or a taper.

### Example 26

The illumination device of Example 25, wherein said taper comprise a tapered light guide.

### Example 27

The illumination device of any of Examples 1-26, further comprising an optical focusing assembly configured to converge the composite light beam and to transmit the composite light beam to a receiving fiber optic conduit or cable.

### Example 28

The illumination device of Example 27, wherein the optical focusing assembly comprises a lens.

### Example 29

The illumination device of any of Examples 1-28, further comprising a fiber optic conduit or cable disposed to receive said composite beam.

### Example 30

The illumination device of any of Examples 1-29, wherein both the first light source and the second light source each comprise a plurality of light emitters.

### Example 31

The illumination device of any of Examples 1-30, further comprising a first plurality of optical fibers optical coupled to said first light source, said first plurality of optical fibers disposed to deliver said first light beam to said first optical combiner.

### Example 32

The illumination device of any of Examples 1-31, further comprising a second plurality of optical fibers optical coupled to said second light source, said second plurality of optical fibers disposed to deliver said second light beam to a second optical combiner.

### Example 33

The illumination device of Example 31 or 32, wherein said plurality of optical fibers comprises a fiber bundle.

### Example 34

The illumination device of any of Examples 31-33, wherein the plurality of optical fibers comprise a first end configured to receive the light flux and a second end configured to emit the received light flux, the plurality of optical fibers at said first end arranged in the first pattern and at the second end arranged in a second pattern different from the first pattern.

### Example 35

The illumination device of Example 34, wherein said first pattern comprises a rectilinear, square or rectangular array.

### Example 36

The illumination device of Example 34 or 35, wherein said second pattern is not a rectilinear, square or rectangular array.

### Example 37

The illumination device of any of Examples 34-36, wherein said second pattern is irregular.

### Example 38

The illumination device of any of Examples 34-37, wherein the fibers in the second pattern have a different (e.g., randomized) order than the fibers in the first pattern.

### Example 39

The illumination device of any of Examples 34-38, wherein said first pattern has a rectilinear, square or rectangular shaped perimeter.

### Example 40

The illumination device of any of Examples 34-39, wherein said second pattern does not have a rectilinear, square or rectangular shaped perimeter.

### Example 41

The illumination device of any of Examples 34-40, wherein said second pattern has a curved, circular, elliptical or oval shaped perimeter.

### Example 42

The illumination device of any of Examples 34-41, wherein said light emitters are arranged in said first pattern.

### Example 43

The illumination device of any of Examples 1-42, wherein the plurality of light sources comprises an array of emitters.

### Example 44

The illumination device of Example 43, wherein the array of emitters comprises a rectilinear, square or rectangular shaped array.

### Example 45

The illumination device of any of Examples 34-44, wherein the first end has a non-round format and the second end has a round format or a different non-round format.

### Example 46

The illumination device of any of Examples 34-45, wherein the first end has a square or rectangular format and second end has a round format.

### Example 47

The illumination device of any of Examples 34-46, wherein the plurality of optical fibers are configured to be moved such that the second ends of the optical fibers are positioned at various selected locations relative to the first ends.

### Example 48

An optical system for imaging a surgical site comprising:
an objective disposed to receive light from a surgical site to provide images of the surgical site;
a light source comprising at least one light emitter;
a light guide having an input at a proximal end and outputs at a plurality of distal ends, said distal ends disposed to provide illumination to said surgical site; and
a plurality of lenses, each lenses disposed with respect to a respective one of said distal ends of said light guide to receive light from said outputs for transmission to said surgical site.

### Example 49

The optical system of Example 48, further comprising at least one camera disposed to receive light from said objective to capture images of said surgical site.

### Example 50

The optical system of Example 48 or 49, wherein said optical system comprises a stereo optical system having first and second, left and right, optical channels.

### Example 51

The optical system of Example 50, wherein said distal ends of said light guide are disposed on opposite sides of said first and second optical channels.

### Example 52

The optical system of Example 50 or 51, wherein said lenses are contained within a circular region having a perimeter defined by outermost edges of said first and second channels.

### Example 53

The optical system of any of Examples 50-52, wherein said optical system comprises first and second cameras for said first and second channels, respectively disposed to receive light from said objective to capture stereo images of said surgical site.

### Example 54

The optical system of any of Examples 48-53, wherein said light source comprises an LED.

### Example 55

The optical system of any of Examples 48-54, wherein said light guide comprise fiber optic light guide.

### Example 56

The optical system of any of Examples 48-54, wherein said light guide comprise a fiber optic bundle.

### Example 57

The optical system of any of Examples 48-54, wherein said light guide comprise a light pipe.

### Example 58

The optical system of any of Examples 48-57, wherein said number of distal ends comprises three distal ends.

### Example 59

The optical system of any of Examples 48-58, wherein said number of distal ends comprises at least four distal ends.

### Example 60

The optical system of any of Examples 48-59, wherein said number of lenses comprises three lenses.

### Example 61

The optical system of any of Examples 48-60, wherein said number of lenses comprises at least four lenses.

### Example 62

The optical system of any of Examples 48-61, wherein said optical system provides a surgical microscope view of the surgical site.

### EXAMPLE SET IV

### Example 1

A distributed surgical lighting system, comprising a user interface configured to control the selection of display views and associated LED sources (light engines) and modes of output from LED sources (e.g., D65, Blue, Green, etc.) for a surgical diagnostic or medically related procedure from a surgical display module, display handle, and control unit console.

### Example 2

A distributed surgical lighting system, comprising: one or more LED groups configured to produce a broadband color spectrum; and configured to be activated prior to display and queried for intensity and color distribution.

### Example 3

A distributed surgical lighting system, comprising: one or more LED groups configured to produce a broadband color spectrum; a processor configured to control the amount of electrical current from a power source to drive each addressable LED source individually within a group of LEDs; and a further timing circuit configured to interrupt the power to all LEDs with in a group of LEDs so that each single LED can be powered and measured by a photodetector, said timing of all LEDs off and one LED on is measured during approximately 1% of a video frame rate.

### Example 4

A distributed surgical lighting system, comprising: a plurality of LED groups (multiple light engines) configured to produce a broadband color spectrum; which can be combined together to form a sufficient intensity and color distribution for a primary display when a selected view would require the camera to depend on its internal adjustments beyond a certain threshold.

### Example 5

A light engine in a distributed surgical lighting system configured to produce a broadband spectrum, comprising: a plurality of LED sources (dies) arranged in a matrix and in proximity to groups of fiber optics or light pipes, and whose output is transformed into a circular form to supply a further fiber optic delivery system to a surgical site.

### Example 6

A light engine in a distributed surgical lighting system configured to produce a broadband spectrum, comprising: a plurality of LED sources (dies) arranged in an assembly of a first source directed towards a group of dichroic mirrors whose axis is 45 degrees to the flow line of illumination, said dichroic mirrors configured to receive the output of further LED sources and direct the combined multiplexed output to a further fiber optic delivery system to a surgical site.

### Example 7

A light engine in a distributed surgical lighting system configured to produce a broadband spectrum as in Example 5 or 6 where the aggregated output of the LEDs is further modified by a tilting plate.

### Example 8

A light engine in a distributed surgical lighting system configured to produce a broadband spectrum as in Example 5 or 6 where the aggregated output of the LEDs is further modified by a tilting plate and a control system utilizing the control circuit and photodetector from Example 3.

### Example 9

A light engine in a distributed surgical lighting system configured to produce a broadband spectrum as in Example 5 or 6 where the aggregated output of the LEDs is modified in combination with a control system where selective LEDs within the broadband spectrum are turned off to produce a blue only, or green only light output.

### Example 10

A distributed surgical lighting system in communication with a user interface and camera control system configured to detect the single color only selections, green or blue, or green and blue, and activate additional light engines capable of producing additional flux for viewing, in which case a different algorithm than the broadband spectrum selection.

### Example 11

A distributed surgical lighting system in communication with a user interface and camera control system configured to detect each camera's status associated with the system and when one or more excitation sources are activated, the cameras without barrier filters are turned off during powering of excitation illumination.

### Example 12

A distributed surgical lighting system, comprising: one or more LED lighting groups where a matrix color aggregation assembly made up of fiber optics assemblies or light pipes can reside in a fiber optic cable.

### Example 13

A distributed surgical lighting system, comprising: one or more LED lighting groups where a matrix color aggregation assembly made up of fiber optics assemblies or light pipes can reside in or on a surgical microscope body.

### Example 14

A distributed surgical lighting system, comprising: one or more LED lighting groups residing in fiber optic cables, surgical microscope bodies, and supplemented via further fiber optics transmitting flux from a remote location, e.g., cart or console, producing supplementary broadband illumination or fluorescence excitation.

### EXAMPLE SET V

### Example 1

A light source comprising rectangular sources, optically coupled to fiber optics or light guides with a similar end face, whose distal end exits to an adjustable element with coatings applied to shift or trim the source output, with further output passing or reflecting by use of a dichroic filter or reflector.

### Example 2

The light source of Example 1, wherein the final output is electrically monitored and adjusted.

### Example 3

A light source comprising one or more circular sources optically coupled to fiber optics or light guides which randomizes and separates into 1/N fibers or light guides for delivery to a surgical site through additional lens elements.

Although described above in connection with particular embodiments, it should be understood the descriptions of the embodiments are illustrative and are not intended to be limiting. Various modifications and applications may occur to those skilled in the art without departing from the true spirit and scope of the invention.

1. An illumination device comprising:
a plurality of light sources, said plurality of light sources including first and second light sources configured to generate first and second light beams respectively;
a first beam combiner configured to receive light from said first and second light sources and combine said first and second light beams to generate a composite light beam; and
a plurality of filters, said plurality of filters comprising a first filter configured to controllably adjust a spectral power distribution of the first light beam incident on the first filter and a second filter configured to controllably adjust a spectral power distribution of the second light beam incident on the second filter,
wherein the first and second filters are configured to be tuned to alter the spectral power distribution of the first and second light beams.

2. The illumination device of claim 1, wherein at least one light source of the plurality of light sources comprises at least one solid-state semiconductor optical emitter producing colored visible light when an electric current is provided.

3. The illumination device of claim 1, wherein the plurality of light sources comprise at least one colored LED.

4. The illumination device of claim 1, wherein the plurality the light sources comprise at least one laser diode.

5. The illumination device of claim 1, wherein at least one source of the plurality of sources comprises at least one solid-state optical emitter containing phosphor and producing a white light output.

6. The illumination device of claim 1, wherein said first beam combiner comprises a reflector that is partially reflective and partially transmissive.

7. The illumination device of claim 1, wherein said first beam combiner comprises a beamsplitter.

8. The illumination device of claim 1, wherein said first beam combiner is partially transmissive, partially reflective surface to transmit said first beam and reflect said second beam.

9. The illumination device of claim 1, wherein said first beam combiner comprises a dichroic reflector configured to transmit light of a first wavelength included in said first light beam and reflect light of a second wavelength included in said second light beam.

10. The illumination device of claim 1, wherein said first light source is a colored light source and said first light beam is a first color and said second light source is a colored light source and said second beam is a second color different from said first color.

11. The illumination device of claim 1, wherein the plurality of light sources further comprises a third light source configured to generate third light beam.

12. The illumination device of claim 11, further comprising a second beam combiner configured to receive light from said third light sources and combine said third light beam with said composite light beam.

13. The illumination device of claim 11, wherein said third light source is a colored light source and said third beam is a third color different from said first and second colors.

14. The illumination device of claim 12, wherein said second beam combiner comprises a reflector that is partially reflective and partially transmissive.

15. The illumination device of claim 12, wherein said second beam combiner comprises a beamsplitter.

16. The illumination device of claim 12, wherein said second beam combiner comprises a dichroic reflector configured to transmit light of a first wavelength included in said first light beam and reflect light of a third wavelength included in said third light beam.

17. The illumination device of claim 12, wherein said second beam combiner is configured to transmit light of a second wavelength included in said second light beam.

18. The illumination device of claim 1, wherein the said first and second optical filters are configured to be tuned by tilting the first and second filters with respect to the respective first and second light beam incident thereon or vice versa.

19. The illumination device of claim 1, wherein the first filter and the second filter each comprises an interference filter.

20. The illumination device of claim 1, wherein the plurality of filters comprises at least one plane-parallel plate with one or both surfaces coated with a thin film coating stack comprising a plurality of layers with different indices of refraction, wherein tilting of the plate relative to the light beam transmitted through the plate selectively alters which wavelengths in the light beam are passed or blocked.

21.-62. (canceled)
